(19) United States
(12) Patent Application Publication
(10) Pub. No.: US 2020/0308582 A1
(43) Pub. Date: Oct. 1, 2020

(54) KCNK3-BASED GENE THERAPY OF CARDIAC ARRHYTHMIA

(71) Applicant: UNIVERSITÄT HEIDELBERG, Heidelberg (DE)

(72) Inventors: Constanze SCHMIDT, Heidelberg (DE); Felix WIEDMANN, Heidelberg (DE); Hugo KATUS, Heidelberg (DE); Dierk THOMAS, Heidelberg (DE); Oliver MÜLLER, Heikendorf (DE)

(21) Appl. No.: 16/754,493

(22) PCT Filed: Oct. 12, 2018
(86) PCT No.: PCT/EP2018/077943

§ 371 Date: Apr. 8, 2020

(30) Foreign Application Priority Data
Oct. 12, 2017 (EP) 17196242.6

(51) Int. Cl. C12N 15/113; C12N 15/86; A61K 48/00; A61P 9/06

(52) U.S. Cl. CPC C12N 15/113; C12N 15/86; A61K 48/0058; A61K 48/0075; C12N 2830/008; A61P 9/06; C12N 2310/14; C12N 2750/14143; C12N 2750/14132; A61K 48/0083

(57) ABSTRACT

The present invention relates to an antagonist of the Two-Pore Domain Potassium Channel (TASK-1) K_{2P}3.1 for use in the prevention and/or treatment of cardiac arrhythmia in a subject. The invention also relates to a nucleic acid molecule usable in the prevention and/or treatment of cardiac arrhythmia in a subject. The invention further relates to a cell comprising said nucleic acid molecule. The invention further relates to a vector comprising said nucleic acid molecule.

The present invention relates to an antagonist of the Two-Pore Domain Potassium Channel (TASK-1) K_{2P}3.1 for use in the prevention and/or treatment of cardiac arrhythmia in a subject in need thereof. The invention also relates to a nucleic acid molecule usable in the prevention and/or treatment of cardiac arrhythmia in a subject. The invention further relates to a cell comprising said nucleic acid molecule. The invention further relates to a vector comprising said nucleic acid molecule.

### BACKGROUND OF THE INVENTION

Atrial fibrillation (AF) is the most common sustained arrhythmia in clinical practice, constituting one of the major causes of stroke, heart failure, and cardiovascular morbidity. In the western world about 2% of the population suffers from paroxysmal, persistent or permanent AF. Prevalence and incidence of AF increase with age, and the number of patients with AF is predicted to rise steeply in our aging population. Despite its epidemiological and individual relevance, current pharmacological, interventional or surgical therapy strategies are limited by suboptimal effectiveness and not uncommonly by severe adverse effects. Until today, safe and effective management of atrial fibrillation remains an unmet medical need. On a molecular level, AF is characterized by structural (i.e. atrial fibrosis, inflammatory infiltrates, enhanced connective tissue deposition, atrial fatty infiltration and amyloid deposition) and electrical remodeling. Rapid ectopic activity may trigger and maintain atrial fibrillation. Moreover, shortening of action potential (AP) duration (APD) is considered a hallmark of atrial remodeling in AF that promotes re-entry, supporting the perpetuation of the arrhythmia. Therefore, suppression of the accelerated atrial repolarization through inhibition of repolarizing K⁺ currents by class III antiarrhythmic drugs represents a pharmacological option for treatment of AF. Over the last decade, research and development in the field of atrial fibrillation were focused on the discovery of atrial specific targets, based on the idea that inhibition of atrial specific targets prevents ventricular and extracardiac side effects. The following ion channels were identified as potential atrial-specific targets: the potassium channels Kv1.5, Kir3.1, Kir3.4 and Kv1.2 as well as the calcium-activated potassium channels. Until today, none of the developed pharmacological compounds targeting these atrial-specific ion channels were brought to clinical application because of low efficiency and several side effects. The family of two-pore-domain (K_{2P}) potassium channels is the youngest (i.e. the least identified) among K⁺ channels. The 15 members of the K_{2P} family are expressed abundantly throughout the body, where they are implicated in several important physiological processes including regulation of cardiac rhythm, mechanical stress, blood pressure, neuroprotection, anesthesia, apoptosis and sensation of oxygen tension, taste or temperature. K_{2P} channels mediate action potential repolarization, and TASK-1 (K_{2P}3.1) currents were recently shown to modulate atrial action potential duration in AF and heart failure (HF). Upregulation of atrial TASK-1 levels in paroxysmal and chronic atrial fibrillation (cAF) contributes to pathological APD shortening. In vitro experiments demonstrated that pharmacological blockade of TASK-1 currents could prolong APD of atrial cardiomyocytes isolated from cAF patients to levels observed among sinus rhythm controls. Cumulative analysis showed, that 30% of the atrial action potential shortening typically observed in AF is explained by the TASK-1 current Inhibition of TASK-1 in cardiomyocytes of chronic atrial fibrillation patients normalizes the APD and thereby prevents the development of atrial reentry circuits. In the human heart expression of TASK-1 subunits is restricted to the atria. Thus, TASK-1 may represent a new atrial specific, mechanism based target for therapy of atrial fibrillation. In comparison to previous atrial specific targets (e.g. Kv1.5) and pharmacological approaches for an atrial-specific AF therapy, interventions targeting the TASK-1 ion channel showed an improved efficiency in normalizing action potential duration. Gene therapy could overcome limitations of traditional pharmacological antiarrhythmic therapy strategies like ventricular proarrhythymic potential, as oligonucleotide-based strategies may provide higher target specificity compared to antiarrhythmic drugs.

The inventors herein present a novel cardiac specific gene therapy, modulating the newly identified atrial specific TASK-1 levels for the treatment or prevention of atrial fibrillation. The developed gene therapy for interfering with TASK-1 expression was tested in a well-established large animal model for atrial fibrillation in pigs. Previous pharmacological compounds and therapeutic approaches for an atrial-selective therapy of AF were mostly tested in healthy mice, despite weaker homology to human patients. Similarly, it was shown that pigs with tachypacing-induced atrial fibrillation, due to consecutively acquired heart failure, could not serve as an animal model with high homology to AF in the human heart. To overcome this limitation, the following steps were conducted: To prevent the consecutive development of heart failure, an AV-node ablation was conducted in pigs before inducing AF. Furthermore, dual chamber pacemakers were implanted to maintain a constant ventricular heart rate during atrial tachypacing. To maintain AF-induction, a biofeedback algorithm was implemented. AF was induced by atrial burst pacing over 20 seconds. Then, AF was consecutively monitored for a certain period to detect AF episodes. If AF occurred in absence of burst pacing, no further burst pacing was applied. Only if sinus rhythm was observed, burst pacing was continued for another period of 20 seconds. By this procedure, continuous atrial fibrillation could be induced without causing heart failure. The inventors observed that this AF model exhibited high homology with AF in the human heart. Subsequently, the inventors successfully tested the newly developed gene therapy in this animal model of AF. Finally, the new gene therapy approach inhibited the expression of TASK-1 and could successfully suppress atrial fibrillation episodes.

### SUMMARY OF THE INVENTION

In a first aspect, the invention relates to an antagonist of the Two-Pore Domain Potassium Channel (TASK-1) K_{2P}3.1 for use in the prevention and/or treatment of cardiac arrhythmia in a subject.

In a second aspect the invention relates to a nucleic acid molecule comprising a polynucleotide, wherein the polynucleotide comprises a nucleotide sequence selected from the group consisting of
(i) at least 10 consecutive nucleotides of the nucleotide sequence according to SEQ ID NO: 1 and 4 and 5 or variants thereof; or
(ii) the RNA encoded by (i); or
(iii) a complement of (i) or (ii).

In a third aspect the invention relates to a vector comprising a nucleic acid molecule of the second aspect of the invention.

In a fourth aspect the invention relates to a cell comprising the nucleic acid according to the second aspect of the invention or the vector of the third aspect of the invention.

### BRIEF DESCRIPTION OF FIGURES

FIG. 1: Schematic diagram of the plasmid pSSV9-siTASK-1-eGFP

Left: The pSSV9-siTASK-1-eGFP plasmid carries a GFP reporter-linked TASK-1 siRNA cassette under control of a cardiomyocyte specific TNT reporter (see FIG. 2 for details). The siRNA section can easily be exchanged by directional cloning using the SacII and BamHI restriction sites. Right: The ITR-flanked dsDNA encoding for the siTASK-1-IRES2-eGFP cassette under control of the cardiomyocyte specific troponin promoter can be packed in AAV6 or AAV9 particles by dual-transfection of pSSV9-siTASK-1-eGFP and the respective REP/CAP plasmid.

FIG. 2: Organization of the ITR-flanked transgene, encoding for pri-miR embedded TASK-1 siRNA

pSSV9-siTASK-1-eGFP carries TASK-1 siRNA, embedded in a pri-miR155 scaffold. For verification of infection efficacy, the reporter protein eGFP is coupled to the siRNA section by an internal ribosome entry site (IRES2). Expression of the siTASK-1-IRES2-eGFP cassette is under control of a cardiomyocyte specific troponin T promoter (TNT i.e. hTNNT2v1). At the bottom: hypothetical secondary structure model of the TASK-1-siRNA carrying miR155 scaffold consisting of conserved miR-flanks, a stem part, carrying the mature TASK-1 siRNA and a terminal loop.

FIG. 3: Species conservation of the siRNA sequences used in this study

A: 3D-structure model of the porcine TASK-1 channel structure, amino acid differences between the human and porcine orthologue are marked in B: siRNA sequences used in this study are compared to human, porcine and rat TASK-1 sequences (see SEQ ID NO: 20 to 28). Nucleotides, conserved over all 3 species are marked with *. C-D: Schematic diagram of the plasmid pSSV9-siTASK-eGFP carrying siRNA against porcine or human TASK-1 (see FIGS. 1 and 2 for details).

FIG. 4: In vivo optimization of the TASK-1 siRNA carrying AAV

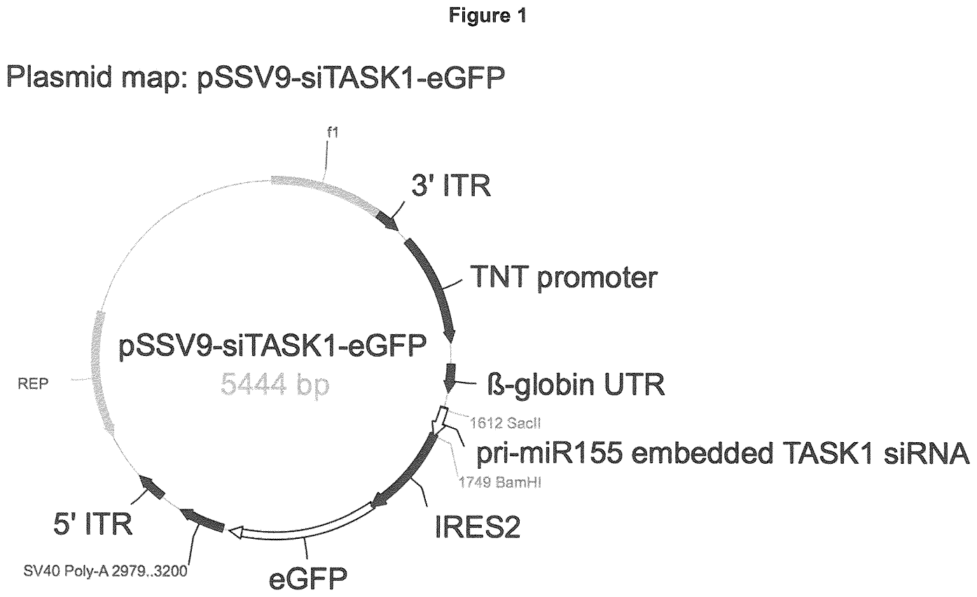

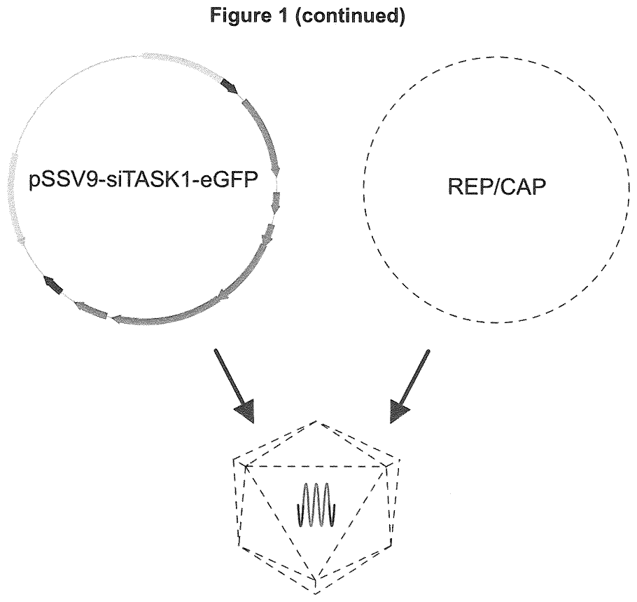

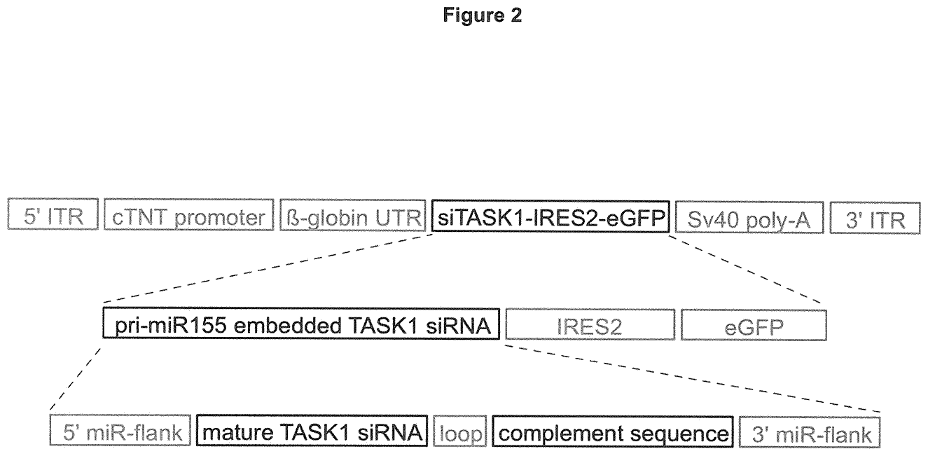

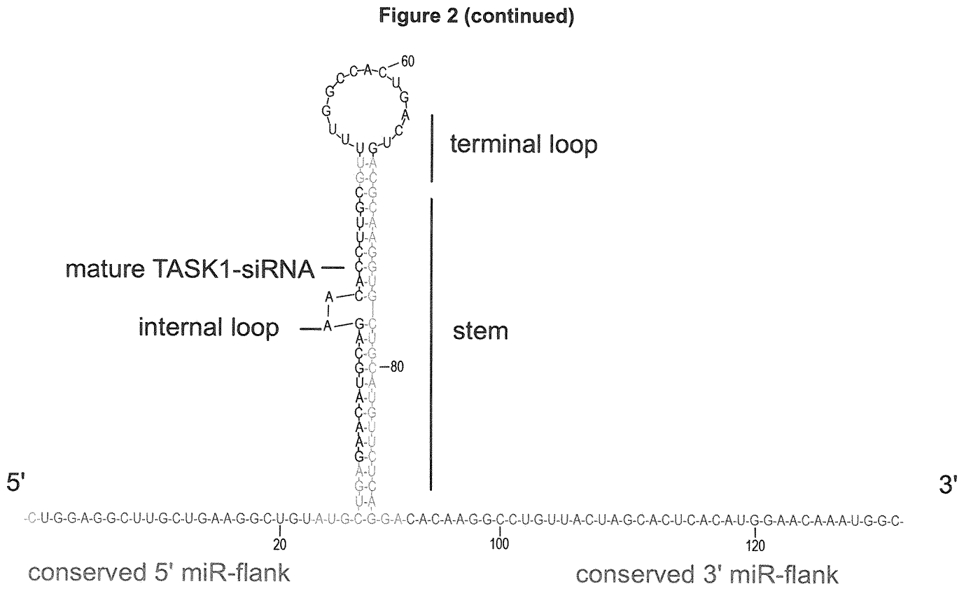

A: Two cardiomyocyte specific promoters, a CMV-enhanced 260-bp myosin light chain (MLC260) promoter and a troponin T (TNT, i.e. hTNNT2v1) were tested for regulation of the miR-siTASK-1-IRES2-eGFP cassette. Comparison of AAV9 production using pSSV9-CMV/MLC260-miR-siTASK-1-IRES2-eGFP and pSSV9-TNT-miR-siTASK-1-IRES2-eGFP shows higher cumulative titers when using the TNT promoter (n=3-5; P<0.0001), therefore constructs using the TNT promoter were used for further studies. B: TASK-1 protein levels of neonatal rat cardiomyocytes infected with AAV6, carrying either scrambled siRNA (siSCRL), or siRNA sequences 1-3. GAPDH protein levels were used as loading control. Highest in vitro efficacy was observed for siRNA3. C: eGFP signal of cultured neonatal rat cardiomyocytes 72 h after infection with AAV6-TNT-siTASK-1-3-eGFP.

FIG. 5: Electrophysiological effects of AAV9-siTASK-1 gene therapy in a large animal model of atrial fibrillation

A: Surface ECG characteristics display no significant changes 14 days after AAV9-siTASK-1-eGFP gene transfer (white bars), when compared to baseline levels (black bars). B-C: sinus node recovery time, measured after programmed stimulation with a basic cycle length of 300-700 ms (SNRT₃₀₀₋₇₀₀) and corrected sinus node recovery times (cSNRT 300-700) show no alteration 14 days after AAV9-siTASK-1-eGFP gene transfer (white bars), when compared to baseline levels (black bars). However, prolonged sinuatrial conduction times (SACT), measured according to Strauss or Narula show significant prolongation 14d after siTASK-1 gene therapy (n=5). According to downregulation of atrial TASK-1 currents, atrial effective refractory periods measured at a basis cycle length of 300, 400 or 500 ms (ARP) display significant prolongation after siTASK-1 gene transfer (n=5). However, ventricular effective refractory periods measured at a basis cycle length of 400 or 500 ms show significant prolongation too (n=5). Electrophysiological characteristics of the atrioventricular node: Ante and retrograde Wenckebach and 2:1 point, AV node effective refractory periods at 300-500 ms (AVNRP₃₀₀₋₅₀₀) basis cycle length and retrograde AVNRP could only be measured under baseline conditions as AV nodal conduction was completely abolished after ablation. * P<0.05, **P<0.001, *** P<0.0001.

FIG. 6: TASK-1 mRNA expression in SR, AF and gene therapy pigs

Right atrial (RA) and left atrial KCNK3 mRNA expression levels, encoding for TASK-1 protein are displayed for sham operated pigs, remaining sinus rhythm (SR), after induction of atrial fibrillation (AF) via right atrial burst pacing for 14 days and for the therapy group, where siTASK-1 gene therapy was applied in pigs suffering from burst pacing induced atrial fibrillation. * P<0.05, **P<0.001, *** P<0.0001 versus the AF group; # P<0.05, ## P<0.001, ### P<0.0001 when comparing SR and the AF-gene therapy group. Data is shown as mean±standard error of the mean, after normalization to mRNA levels of the housekeeping gene importin 8 (IPO8).

FIG. 7: Atrial TASK-1 protein expression in pigs suffering from atrial fibrillation compared to individuals receiving gene therapy

TASK-1 protein levels, detected via immunoblot (see inlays) are shown for samples from porcine left atria (LA), right atria (RA), left atrial appendages (LAA) and right atria appendages (RAA). A-D: Comparison of sham operated animals, remaining in sinus rhythm (SR) burst pacing induced atrial fibrillation (AF), either sham treated with AAV9-eGFP or receiving anti TASK-1 gene therapy by injection of AAV9-siTASK-1-eGFP. * P<0.05, **P<0.001, *** P<0.0001 versus the AF group; # P<0.05, ## P<0.001, ### P<0.0001 when comparing SR and the AF-gene therapy group. Data is shown as mean of 5 individual animals in each group±standard error of the mean, after normalization to protein levels of the housekeeping gene glycerinaldehyd-3-phosphat-dehydrogenase (GAPDH).

FIG. 8: Comparison of TASK-1 currents in atrial cardiomyocytes, isolated from AF, SR and gene therapy pigs

TASK-1 current densities of single atrial cardiomyocytes from different study groups are shown: SR, sinus rhythm without gene transfer; AF, induction of atrial fibrillation without gene transfer; si-SR, antiTASK-1 gene therapy in SR, si-AF: anti TASK-1 gene therapy in AF. Data is depicted as mean±standard error of the mean, after normalization to cell capacity in pF. * P<0.05, **P<0.001, *** P<0.0001.

FIG. 9: AF, SR and gene therapy pigs exhibit different atrial action potential durations

Action potential duration, measured at 50% (APD₅₀) or 90% (APD₉₀) of repolarization were measured via patch-clamp technique in the current clamp configuration on isolated atrial cardiomyocytes from the following study groups: SR, sinus rhythm without gene transfer; AF, induction of atrial fibrillation without gene transfer; si-SR, antiTASK-1 gene therapy in SR, si-AF: anti TASK-1 gene therapy in AF. Data is depicted as mean±standard error of the mean. * P<0.05, **P<0.001, *** P<0.0001.

### LIST OF SEQUENCES

SEQ ID NO: 1 si-p/hTASK-1-1: DNA sequence corresponding to the siRNA sequence 1 directed against the porcine and the human orthologue of TASK-1.
SEQ ID NO: 2 si-pTASK-1-2: DNA sequence corresponding to the siRNA sequence 2 directed against the porcine orthologue of TASK-1.
SEQ ID NO: 3 si-pTASK-1-3: DNA sequence corresponding to the siRNA sequence 3 directed against the porcine orthologue of TASK-1.
SEQ ID NO: 4 si-hTASK-1-2: DNA sequence corresponding to the siRNA sequence 2 directed against the human orthologue of TASK-1.
SEQ ID NO: 5 si-hTASK-1-3: DNA sequence corresponding to the siRNA sequence 3 directed against the human orthologue of TASK-1.
SEQ ID NO: 6 pSSV9-TNT-miR/si-p/hTASK-1-1-IRES2-eGFP: Plasmid carrying the ITR-flanked construct of TASK-1 siRNA sequence 1 (directed against the porcine and the human TASK-1 orthologue) coupled to an eGFP-Reporter. via IRES2 under control of the cardiomyocyte specific troponin promoter
SEQ ID NO: 7 pSSV9-TNT-miR/si-pTASK-1-2-IRES2-eGFP: Plasmid carrying the ITR-flanked construct of TASK-1 siRNA sequence 2 (directed against the porcine TASK-1 orthologue) coupled to an eGFP-Reporter via IRES2 under control of the cardiomyocyte specific troponin promoter.
SEQ ID NO: 8 pSSV9-TNT-miR/si-pTASK-1-3-IRES2-eGFP: Plasmid carrying the ITR-flanked construct of TASK-1 siRNA sequence 3 (directed against the porcine TASK-1 orthologue) coupled to an eGFP-Reporter via IRES2 under control of the cardiomyocyte specific troponin promoter.
SEQ ID NO: 9 pSSV9-TNT-miR/si-hTASK-1-2-IRES2-eGFP: Plasmid carrying the ITR-flanked construct of TASK-1 siRNA sequence 2 (directed against the human TASK-1 orthologue) coupled to an eGFP-Reporter via IRES2 under control of the cardiomyocyte specific troponin promoter.
SEQ ID NO: 10 pSSV9-TNT-miR/si-hTASK-1-3-IRES2-eGFP: Plasmid carrying the ITR-flanked construct of TASK-1 siRNA sequence 3 (directed against the human TASK-1 orthologue) coupled to an eGFP-Reporter via IRES2 under control of the cardiomyocyte specific troponin promoter.
SEQ ID NO: 11 pSSV9-CMV/MLC-miR/si-p/hTASK-1-1-IRES2-eGFP: Plasmid carrying the ITR-flanked construct of TASK-1 siRNA sequence 1 (directed against the porcine and the human TASK-1 orthologue) coupled to an eGFP-Reporter via IRES2 under control of the cardiomyocyte specific CMV-enhanced MLC260 promoter.
SEQ ID NO: 12 pSSV9-CMV/MLC-miR/si-pTASK-1-2-IRES2-eGFP: Plasmid carrying the ITR-flanked construct of TASK-1 siRNA sequence 2 (directed against the porcine TASK-1 orthologue) coupled to an eGFP-Reporter via IRES2 under control of the cardiomyocyte specific CMV-enhanced MLC260 promoter.
SEQ ID NO: 13 pSSV9-CMV/MLC-miR/si-pTASK-1-3-IRES2-eGFP: Plasmid carrying the ITR-flanked construct of TASK-1 siRNA sequence 3 (directed against the porcine TASK-1 ortholog) coupled to an eGFP-Reporter via IRES2 under control of the cardiomyocyte specific CMV-enhanced MLC260 promoter.
SEQ ID NO: 14 pSSV9-CMV/MLC-miR/si-hTASK-1-2-IRES2-eGFP: Plasmid carrying the ITR-flanked construct of TASK-1 siRNA sequence 2 (directed against the human TASK-1 ortholog) coupled to an eGFP-Reporter via IRES2 under control of the cardiomyocyte specific CMV-enhanced MLC260 promoter.
SEQ ID NO: 15 pSSV9-CMV/MLC-miR/si-hTASK-1-3-IRES2-eGFP: Plasmid carrying the ITR-flanked construct of TASK-1 siRNA sequence (directed against the human TASK-1 ortholog) coupled to an eGFP-Reporter via IRES2 under control of the cardiomyocyte specific CMV-enhanced MLC260 promoter.
SEQ ID NO: 16 hTASK-1 siRNA from Hao and Li (J Mol Neurosci. 2015 55:314-7).
SEQ ID NO: 17 hTASK-1 siRNA from Olschewski et al. (Circ Res. 2006 98:1072-80).
SEQ ID NO: 18 hTASK-1 siRNA from Gurney and Hunter (J Pharmacol Toxicol Met 51:253-62).
SEQ ID NO: 19 hTASK-1 siRNA from Tang et al. (Am J Respir Cell Mol Biol. 41:476-83).
SEQ ID NO: 20 human TASK-1 sequence; transcript variant X1, coding sequence used for design of siRNA 1.
SEQ ID NO: 21 rat TASK-1 sequence, coding sequence used for design of siRNA 1.
SEQ ID NO: 22 porcine TASK-1 sequence, coding sequence used for design of siRNA 1.
SEQ ID NO: 23 human TASK-1 sequence; transcript variant X1, coding sequence used for design of siRNA 2.
SEQ ID NO: 24 rat TASK-1 sequence, coding sequence used for design of siRNA 2.
SEQ ID NO: 25 porcine TASK-1 sequence, coding sequence used for design of siRNA 2.
SEQ ID NO: 26 human TASK-1 sequence; transcript variant X1, coding sequence used for design of siRNA 3.
SEQ ID NO: 27 rat TASK-1 sequence, coding sequence used for design of siRNA 3.
SEQ ID NO: 28 porcine TASK-1 sequence, coding sequence used for design of siRNA 3.

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

All sequences referred to herein are disclosed in the attached sequence listing that, with its whole content and disclosure, is a part of this specification.

As used herein, an “antagonist” refers to a compound, drug or molecule that interlocks or disables a biological response caused by the interaction partner of the antagonist.

The terms “inhibition”, “inhibiting” or “inhibitory” are used interchangeably and relate to a molecule that decreases or prevents a chemical or biological reaction.

The term “Two-Pore Domain Potassium Channel” as used herein refers to the two pore domain potassium channel subfamily K member 3, KCNK3, K_{2P}3.1, TASK-1. These channels are regulated by several mechanisms including oxygen tension, pH, mechanical stretch, and G-proteins.

As used herein, “treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in subject.

The term “cardiac arrythmia” as used herein refers to a group of conditions in which the heartbeat is irregular, either too fast, or too slow. There exist four main types of arrhythmia: extra beats, supraventricular tachycardias, ventricular arrhythmias, and bradyarrhythmias. Extra beats include premature atrial contractions, premature ventricular contractions, and premature junctional contractions. Supraventricular tachycardias include atrial fibrillation, atrial flutter, and paroxysmal supraventricular tachycardia. Ventricular arrhythmias include ventricular fibrillation and ventricular tachycardia.

As used herein, a “subject” means any mammal or bird who may benefit from a treatment with the antagonist described herein (i.e. with an antagonist of the Two-Pore Domain Potassium Channel (TASK-1) K_{2P}3.1). Preferably, a “subject” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including chimpanzees and human beings. It is particularly preferred that the “subject” is a human being.

The terms “polynucleotide” and “nucleic acid” are used interchangeably herein and are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA), deoxyribonucleic acid (DNA), and mixtures thereof such as e.g. RNA-DNA hybrids (within one strand), as well as cDNA, genomic DNA, recombinant DNA, cRNA and mRNA. A nucleic acid may consist of an entire gene, or a portion thereof, the nucleic acid may also be a miRNA, siRNA, or a piRNA. MiRNAs are short ribonucleic acid (RNA) molecules, which are on average 22 nucleotides long but may be longer and which are found in all eukaryotic cells, i.e. in plants, animals, and some viruses, which functions in transcriptional and post-transcriptional regulation of gene expression. MiRNAs are post-transcriptional regulators that bind to complementary sequences on target messenger RNA transcripts (mRNAs), usually resulting in translational repression and gene silencing. MiRNAs comprise microRNA sponges, anti-miRNA oligonucleotides, chemically modified miRNA mimics, pre-miRNA, pri-miRNA, anti-pre-miRNA oligonucleotides, anti-pri-miRNA oligonucleotides. Small interfering RNAs (siRNAs), sometimes known as short interfering RNA or silencing RNA, are short ribonucleic acid (RNA molecules), between 20-25 nucleotides in length. They are involved in the RNA interference (RNAi) pathway, where they interfere with the expression of specific genes. PiRNAs are also short RNAs which usually comprise 26-31 nucleotides and derive their name from so-called piwi proteins they are binding to. The nucleic acid can also be an artificial nucleic acid. Artificial nucleic acids include polyamide or peptide nucleic acid (PNA), morpholino and locked nucleic acid (LNA), as well as glycol nucleic acid (GNA) and threose nucleic acid (TNA). Each of these is distinguished from naturally-occurring DNA or RNA by changes to the backbone of the molecule. The nucleic acids, can e.g. be synthesized chemically, e.g. in accordance with the phosphotriester method (see, for example, Uhlmann, E. & Peyman, A. (1990) Chemical Reviews, 90, 543-584). In the context of the present invention the term “nucleic acid” includes but is not limited

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility.

The term “ligand” as used herein can include naturally occurring molecules, or recombinant or synthetic molecules. Non-limiting examples of a ligand can include a cell surface receptor ligand, a targeting ligand, an antibody or a portion thereof, an antibody-like molecule, an enzyme, an antigen, an active agent, a small molecule, a protein, a peptide, a peptidomimetic, a carbohydrate (e.g., but not limited to, monosaccharides, disaccharides, trisaccharides, oligosaccharides, polysaccharides, and lipopolysaccharides), an aptamer, a cytokine, a lectin, a lipid, a plasma albumin, and any combinations thereof. As used herein, the term “ligand” refers to a molecule that binds to or interacts with a target molecule. Typically the nature of the interaction or binding is noncovalent, e.g., by hydrogen, electrostatic, or van der Waals interactions, however, binding can also be covalent.

As used in this specification the term “vector”, also referred to as an expression construct, is usually a plasmid or virus designed for protein expression in cells. The term “vector” refers to a protein or a polynucleotide or a mixture thereof which is capable of being introduced or of introducing proteins and/or nucleic acids comprised therein into a cell. Examples of vectors include but are not limited to plasmids, cosmids, phages, viruses or artificial chromosomes. In particular, a vector is used to transport a gene product of interest, such as e.g. foreign or heterologous DNA into a suitable host cell. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as but not limited to promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be control by a separate expression control sequence or all polynucleotides comprised on the vector may be controlled by a single expression control sequence. Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “AAV” (adeno associated virus) as used in the context of the present invention refers to a complete virus particle, such as a wild-type (“wt”) AAV virus particle (i.e., including a linear, single-stranded AAV nucleic acid genome associated with an AAV capsid protein coat). In this regard, single-stranded AAV nucleic acid molecules of either complementary sense (i.e., “sense” or “antisense” strands) can be packaged into any one AAV virion; both strands are equally infectious. An AAV vector of the present invention may be produced in a suitable host cell which has had an AAV vector, AAV helper functions and accessory functions introduced therein. In this manner, the host cell is rendered capable of encoding AAV polypeptides that are required for packaging the AAV genome (i.e., containing a recombinant nucleotide sequence of interest) into recombinant virion particles for subsequent gene delivery.

The term “expression control sequence” as used herein refers to nucleotide sequence which controls expression of a target gene linked downstream of the expression control sequence.

The term “promoter” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

The term “tissue-specific promoter” as used in the context of the present invention means a promoter which mediates transcription of the downstream gene only in a particular tissue. Use of the tissue-specific promoter allows a protein or a functional RNA to be expressed tissue-specifically, for example in heart tissue.

“Operably linked” as used in the context of the present invention refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

An “effective amount” or “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, the term “variant” is to be understood as a polynucleotide which differs in comparison to the polynucleotide from which it is derived by one or more changes in its length or sequence. The polynucleotide from which a polynucleotide variant is derived is also known as the parent polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent polynucleotide is a wild-type polynucleotide, or a consensus sequence thereof. However, also naturally occurring variants are to be understood to be encompassed by the term “variant” as used herein. Further, the variants usable in the present invention may also be derived from homologs, orthologues, or paralogs of the parent molecule or from artificially constructed variant, provided that the variant exhibits at least one biological activity of the parent molecule, i.e. is functionally active.

Alternatively, or additionally, a “variant” as used herein, can be characterized by a certain degree of sequence identity to the polynucleotide from which it is derived. More precisely, a polypeptide variant in the context of the present invention exhibits at least 80% sequence identity to its parent polynucleotide. The sequence identity polynucleotide variant is over a continuous stretch of 5, 10, 15, 20, 25, 30, 40, 45, 50, 60, 70, 80, 90, 100 or more nucleotides.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity.

The term “identical” in the context of two or more nucleic acids or polypeptide sequences, refers to two or more sequences or sub sequences that are the same, i.e. comprise the same sequence of nucleotides. Sequences are “substantially identical” to each other if they have a specified percentage of nucleotides residues that are the same (e.g., at least 20%, at least 25%, at least 30%, at least 35%, at least 40%, at least 45%, at least 50%, at least 55%, at least 60%, at least 65%, at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over a specified region), when compared and aligned for maximum correspondence over a comparison window, or designated region as measured using one of the following sequence comparison algorithms or by manual alignment and visual inspection. These definitions also refer to the complement of a test sequence. Accordingly, the term “at least 80% sequence identity” is used throughout the specification with regard to polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In the human heart expression of TASK-1 subunits is predominantly restricted to the atria. Thus, TASK-1 K_{2P}3.1 may represent an atrial specific mechanism based target for therapy of atrial fibrillation. The inventors could surprisingly show that gene therapy could overcome limitations of traditional pharmacological antiarrhythmic therapy strategies like ventricular proarrhythymic potential, as oligonucleotide-based strategies may provide higher target specificity compared to antiarrhythmic drugs. The inventors herewith present a novel cardiac specific gene therapy, modulating atrial TASK-1 K_{2P}3.1 levels for the treatment or prevention of atrial fibrillation.

This surprising finding provides inter alia the following advantages over the art: (i) highly target specific gene therapy approach in comparison to conventional therapy with antiarrhythmic drugs, (ii) reduction of adverse side effects resulting in saver therapy (iii) effective treatment or prevention of atrial fibrillation, (iii) less frequent administration of therapy leading to less therapeutic burden of the subject; (iv) prolonged therapy efficacy due to gene therapeutic approach; (v) physiological/mechanism based therapy approach, targeting the arrhythmogenic substrate of AF (i.e. TASK-1 upregulation).

In a first aspect, the present invention provides an antagonist of TASK-1 for use in the prevention and/or treatment of cardiac arrhythmia in a subject.

In a preferred embodiment of the first aspect the antagonist inhibits translation of TASK-1 K_{2P}3.1 encoding mRNA. Preferably, the inhibitor of translation of TASK-1 K_{2P}3.1 encoding mRNA is selected from the group consisting of inhibitory nucleic acids, e.g. siRNA or shRNA, miRNA or lncRNA, microRNA-sponges, anti-miRNA oligonucleotides, chemically modified miRNA mimics, pre-miRNA, pri-miRNA, anti-pre-miRNA oligonucleotides, anti-pri-miRNA oligonucleotides, or derivatives thereof. More preferably the inhibitory nucleic acid is comprised in a microRNA precursor or derivatives thereof. Even more preferably, the inhibitory nucleic acid is comprised in a pri-miRNA scaffold. It is even more preferred that the pri-miRNA scaffold is the pri-miR155 scaffold, the pri-miR1 scaffold, the pri-miR30 scaffold, the pri-miR125b scaffold, or the pri-miR150 scaffold. Even more preferably the scaffold is the pri-miR155 scaffold.

In a preferred embodiment the antagonist for use in the prevention and/or treatment of cardiac arrhythmia in a subject is a nucleic acid molecule. The nucleic acid molecule comprises a polynucleotide, wherein the polynucleotide comprises a nucleotide sequence selected from the group consisting of:
(i) at least 10 consecutive nucleotides of the nucleotide sequence according to SEQ ID NO: 1, SEQ ID NO: 4, SEQ ID NO: 5, SEQ ID NO: 16, SEQ ID NO: 17, SEQ ID NO: 18, SEQ ID NO: 19 or variants thereof; or
(ii) the RNA encoded by (i); or
(iii) a complement (i) or (ii).

In a preferred embodiment the nucleotide sequence comprises at least 10, more preferably at last 15, more preferably at least 20 consecutive nucleotides of the nucleotide sequence according to SEQ ID NO: 1 and most preferably the entire nucleotide sequence according to SEQ ID NO: 1. In a more preferred embodiment the nucleotide sequence comprises at least 10, more preferably at last 15, more preferably at least 20 consecutive nucleotides of the nucleotide sequence according to SEQ ID NO: 4 and most preferably the entire nucleotide sequence according to SEQ ID NO: 4. In an even more preferred embodiment the nucleotide sequence comprises at least 10, more preferably at last 15, more preferably at least 20 consecutive nucleotides of the nucleotide sequence according to SEQ ID NO: 5 and most preferably the entire nucleotide sequence according to SEQ ID NO: 5. In a preferred embodiment the nucleotide sequence comprises at least 10, more preferably at last 15, more preferably at least 20 consecutive nucleotides of the nucleotide sequence according to SEQ ID NO: 16 and most preferably the entire nucleotide sequence according to SEQ ID NO: 16. In a preferred embodiment the nucleotide sequence comprises at least 10, more preferably at least 15, more preferably at least 20 consecutive nucleotides of the nucleotide sequence according to SEQ ID NO: 17 and most preferably the entire nucleotide sequence according to SEQ ID NO: 17. In a preferred embodiment the nucleotide sequence comprises at least 10, more preferably at last 15, more preferably at least 20 consecutive nucleotides of the nucleotide sequence according to SEQ ID NO: 18 and most preferably the entire nucleotide sequence according to SEQ ID NO: 18. In a preferred embodiment the nucleotide sequence comprises at least 10, more preferably at last 15, more preferably at least 20 consecutive nucleotides of the nucleotide sequence according to SEQ ID NO: 19 and most preferably the entire nucleotide sequence according to SEQ ID NO: 19.

In another preferred embodiment that nucleotide sequence comprises at least 10 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 10 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 10 nucleotides of a variant of the nucleotide sequence according to SEQ ID NO: 5, wherein the variants have at least 85%, 90%, 95% or 99% sequence identity to SEQ ID NO: 5. In another preferred embodiment that nucleotide sequence comprises at least 10 nucleotides of a variant of the nucleotide sequence according to SEQ ID NO: 16, wherein the variants have at least 85%, 90%, 95% or 99% sequence identity to SEQ ID NO: 16. In another preferred embodiment that nucleotide sequence comprises at least 10 nucleotides of a variant of the nucleotide sequence according to SEQ ID NO: 17, wherein the variants have at least 85%, 90%, 95% or 99% sequence identity to SEQ ID NO: 17. In another preferred embodiment that nucleotide sequence comprises at least 10 nucleotides of a variant of the nucleotide sequence according to SEQ ID NO: 18, wherein the variants have at least 85%, 90%, 95% or 99% sequence identity to SEQ ID NO: 18. In another preferred embodiment that nucleotide sequence comprises at least 10 nucleotides of a variant of the nucleotide sequence according to SEQ ID NO: 19, wherein the variants have at least 85%, 90%, 95% or 99% sequence identity to SEQ ID NO: 19.

In another preferred embodiment that nucleotide sequence comprises at least 15 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 15 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 15 nucleotides of a variant of the nucleotide sequence according to SEQ ID NO: 5, wherein the variants have at least 85%, 90%, 95% or 99% sequence identity to SEQ ID NO: 5. In another preferred embodiment that nucleotide sequence comprises at least 15 nucleotides of a variant of the nucleotide sequence according to SEQ ID NO: 16, wherein the variants have at least 85%, 90%, 95% or 99% sequence identity to SEQ ID NO: 16. In another preferred embodiment that nucleotide sequence comprises at least 15 nucleotides of a variant of the nucleotide sequence according to SEQ ID NO: 17, wherein the variants have at least 85%, 90%, 95% or 99% sequence identity to SEQ ID NO: 17. In another preferred embodiment that nucleotide sequence comprises at least 15 nucleotides of a variant of the nucleotide sequence according to SEQ ID NO: 18, wherein the variants have at least 85%, 90%, 95% or 99% sequence identity to SEQ ID NO: 18. In another preferred embodiment that nucleotide sequence comprises at least 15 nucleotides of a variant of the nucleotide sequence according to SEQ ID NO: 19, wherein the variants have at least 85%, 90%, 95% or 99% sequence identity to SEQ ID NO: 19.

In another preferred embodiment that nucleotide sequence comprises at least 20 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 20 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 20 nucleotides of a variant of the nucleotide sequence according to SEQ ID NO: 5, wherein the variants have at least 85%, 90%, 95% or 99% sequence identity to SEQ ID NO: 5. In another preferred embodiment that nucleotide sequence comprises at least 20 nucleotides of a variant of the nucleotide sequence according to SEQ ID NO: 16, wherein the variants have at least 85%, 90%, 95% or 99% sequence identity to SEQ ID NO: 16. In another preferred embodiment that nucleotide sequence comprises at least 20 nucleotides of a variant of the nucleotide sequence according to SEQ ID NO: 17, wherein the variants have at least 85%, 90%, 95% or 99% sequence identity to SEQ ID NO: 17. In another preferred embodiment that nucleotide sequence comprises at least 20 nucleotides of a variant of the nucleotide sequence according to SEQ ID NO: 18, wherein the variants have at least 85%, 90%, 95% or 99% sequence identity to SEQ ID NO: 18. In another preferred embodiment that nucleotide sequence comprises at least 20 nucleotides of a variant of the nucleotide sequence according to SEQ ID NO: 19, wherein the variants have at least 85%, 90%, 95% or 99% sequence identity to SEQ ID NO: 19.

In another preferred embodiment that nucleotide sequence is a variant of the nucleotide sequence according to SEQ ID NO: 1, wherein the variants have at least 85%, 90%, 95% or 99% sequence identity to SEQ ID NO: 1. In another preferred embodiment that nucleotide sequence is a variant of the nucleotide sequence according to SEQ ID NO: 4, wherein the variants have at least 85%, 90%, 95% or 99% sequence identity to SEQ ID NO: 4. In another preferred embodiment that nucleotide sequence is a variant of the nucleotide sequence according to SEQ ID NO: 5, wherein the variants have at least 85%, 90%, 95% or 99% sequence identity to SEQ ID NO: 5. In another preferred embodiment that nucleotide sequence is a variant of the nucleotide sequence according to SEQ ID NO: 16, wherein the variants have at least 85%, 90%, 95% or 99% sequence identity to SEQ ID NO: 16. In another preferred embodiment that nucleotide sequence is a variant of the nucleotide sequence according to SEQ ID NO: 17, wherein the variants have at least 85%, 90%, 95% or 99% sequence identity to SEQ ID NO: 17. In another preferred embodiment that nucleotide sequence is a variant of the nucleotide sequence according to SEQ ID NO: 18, wherein the variants have at least 85%, 90%, 95% or 99% sequence identity to SEQ ID NO: 18. In another preferred embodiment that nucleotide sequence is a variant of the nucleotide sequence according to SEQ ID NO: 19, wherein the variants have at least 85%, 90%, 95% or 99% sequence identity to SEQ ID NO: 19.

In each of the above cases the nucleic acid is alternatively the RNA encoded by the respective nucleic acid or the complement of the nucleic acid or RNA.

It is preferred that the nucleic acid sequences as described above and according to the SEQ IDs above further comprise ITR sequences.

In another embodiment the antagonist inhibits transcription of TASK-1 encoding mRNA or the processing thereof. Preferably, the inhibitor of transcription of TASK-1 is selected from the group consisting of oligonucleotides, proteins or compositions thereof, modifying methylation of genomic DNA, folding of genomic DNA and histone phosphorylation or the accessibility of translation initiators, enhancers or genomic DNA encoding for TASK-1 mRNA.

In another preferred embodiment the antagonist inhibits maturation, post-translational modification, trafficking, recycling, or degradation or activity of TASK-1. Preferably, the inhibitor of maturation, post-translational modification, trafficking, recycling, or degradation or activity of TASK-1 is selected from the group consisting of N-glycosylation inhibitor tunicamycin, 14-3-3 inhibitor 2-(2,3-Dihydro-1,5-dimethyl-3-oxo-2-phenyl-1H-pyrazol-4-yl)-2,3-dihydro-1,3-dioxo-1H-isoindole-5-carboxylicacid, siRNA downregulating P11 or β-COP.

In another preferred embodiment the antagonist inhibits the function of TASK-1 K_{2P}3.1. Preferably, the inhibitor of the function of TASK-1 K_{2P}3.1 is selected from the group consisting of a ligand specifically binding to TASK-1 K_{2P}3.1, a nucleic acid encoding such a ligand, a protein or compound increasing phosphorylation of TASK-1 K_{2P}3.1. In some embodiments, a ligand can include an active agent which refers to a molecule that is to be delivered to a cell or to a target area. Accordingly, without limitation, an active agent can be selected from the group consisting of small organic or inorganic molecules, plasmids, vectors, monosaccharides, disaccharides, trisaccharides, oligosaccharides, polysaccharides, biological macromolecules, e.g., peptides, proteins, peptide analogs and derivatives thereof, peptidomimetics, nucleic acids (e.g., but not limited to, DNA, RNA, mRNA, tRNA, RNAi, siRNA, microRNA, or any other art-recognized RNA or RNA-like molecules), nucleic acid analogs and derivatives, polynucleotides, oligonucleotides, enzymes, antibiotics, an extract made from biological materials such as bacteria, plants, fungi, or animal cells or tissues, naturally occurring or synthetic compositions, therapeutic agents, preventative agents, diagnostic agents, imaging agents, antibodies or portions thereof, antibody-like molecules, aptamers (e.g., nucleic acid or protein aptamers) or any combinations thereof. Even more preferably, the protein or compound modulating phosphorylation of TASK-1 is selected from the group consisting of endothelin-1, platelet activating factor, the PKCε activator CRACK, serotonin, thyrotropin releasing hormone (TRH), acetylcholine, angiotensin II or the α1-adrenergic agonist methoxamine.

In another preferred embodiment the inhibitory nucleic acid is comprised in a vector. Preferably, the vector is selected from the group consisting of plasmid vectors, cosmid vectors or viral vectors. The term viral vector encompasses not only viral vectors that are modified to carry a transgene of interest but also those viral vectors that are modified to improve their half-life in the serum or to target them to cells of a particular tissue. Preferred viral vectors are modified to have a tropism to heart tissue in particular to cardiomyocytes. This may be achieved by modifying envelope and/or coat proteins of the viral vector in such that ligands are exposed on the surface of the viral vector that specifically bind to a receptor that is present on heart tissue in particular on cardiomyocytes. It is preferred that the viral vector is selected from the group consisting of an adenoviral vector, AAV vector, alphaviral vector, herpes viral vector, measles viral vector, pox viral vector, vesicular stomatitis viral vector, retroviral vector and lentiviral vector or phage vector. Even more preferably the viral vector is an AAV vector. Even more preferably, the AVV vector is selected from the group consisting of AAV1, AAV2, AAV3, AAV4, AAV5, AAV6, AAV7, AAV8, AAV9, AAV10, AAVrh10, AAV11, and AAV12 or a variant thereof. Even more preferably the vector is an AAV2, AAV6 or AAV9. Most preferably the vector is an AAV9 vector and/or a variant of AAV9 with an altered tropism to heart tissue, i.e. the coat protein of the AAV9 variant is modified to specifically target heart tissue. It is preferred that these variants specifically target cardiomyocytes. More preferably the AAV9 variants target heart cells. Even more preferably, the AAV9 variants target atrial cells

In another embodiment it is preferred that the inhibitory nucleic acid is operably linked to an expression control sequence. Preferably, the expression control sequence is a heart tissue specific promoter. More preferably, the heart-tissue specific promoter is selected from the group consisting of Cardiac Actin Enhancer/Elongation Factor 1 promoter, Cytomegali-virus enhancer/Myosin light chain ventricle 2 promoter, troponin, Atrial Natriuretic Peptide or Slow Myosin Heavy Chain 3 Gene. Even more preferably, the heart-tissue specific promoter is troponin.

It is also preferred that the antagonist of TASK-1K_{2P}3.1 for use in the treatment and/or prevention of cardiac arrhythmia is comprised in a pharmaceutical composition. The pharmaceutical composition comprises an effective amount or therapeutically effective amount of the antagonist of TASK-1. The pharmaceutical compositions can take the form of solutions, suspensions, emulsion, tablets, pills, capsules, powders, sustained-release formulations and the like. The pharmaceutical composition can be formulated as a suppository, with traditional binders and carriers such as triglycerides.

For preparing pharmaceutical compositions of the present invention, pharmaceutically acceptable carriers can be either solid or liquid. Solid form compositions include powders, tablets, pills, capsules, lozenges, cachets, suppositories, and dispersible granules. A solid excipient can be one or more substances, which may also act as diluents, flavoring agents, binders, preservatives, tablet disintegrating agents, or an encapsulating material. In powders, the excipient is preferably a finely divided solid, which is in a mixture with the finely divided inhibitor of the present invention. In tablets, the active ingredient is mixed with the carrier having the necessary binding properties in suitable proportions and compacted in the shape and size desired. Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. For preparing suppositories, a low melting wax, such as a mixture of fatty acid glycerides or cocoa butter, is first melted and the active component is dispersed homogeneously therein, as by stirring. The molten homogeneous mixture is then poured into convenient sized moulds, allowed to cool, and thereby to solidify. Tablets, powders, capsules, pills, cachets, and lozenges can be used as solid dosage forms suitable for oral administration.

Liquid form compositions include solutions, suspensions, and emulsions, for example, water, saline solutions, aqueous dextrose, glycerol solutions or water/propylene glycol solutions. For parenteral injections (e.g. intravenous, intraarterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is administered intravenously.

In particular embodiments, the pharmaceutical composition is in unit dosage form. In such form the composition may be subdivided into unit doses containing appropriate quantities of the active component. The unit dosage form can be a packaged composition, the package containing discrete quantities of the composition, such as packaged tablets, capsules, and powders in vials or ampoules. Also, the unit dosage form can be a capsule, an injection vial, a tablet, a cachet, or a lozenge itself, or it can be the appropriate number of any of these in packaged form.

The composition, if desired, can also contain minor amounts of wetting or emulsifying agents, or pH buffering agents.

Furthermore, such pharmaceutical composition may also comprise other pharmacologically active substance such as but not limited to adjuvants and/or additional active ingredients. Adjuvants in the context of the present invention include but are not limited to inorganic adjuvants, organic adjuvants, oil-based adjuvants, cytokines, particulate adjuvants, virosomes, bacterial adjuvants, synthetic adjuvants, or synthetic polynucleotides adjuvants.

In another preferred embodiment the antagonist of TASK-1 K_{2P}3.1 or the pharmaceutical composition for use in the prevention and/or treatment of cardiac arrhythmia is administered peroral, inhalative, by intravenous, intramucosal, intraarterial, intramusculuar, intracardiac, intraatrial or intracoronal injection, more preferably intraatrial.

In another preferred embodiment the viral vector is administered in a dosage of 1×10¹¹-1×10¹⁴ viral particles per dose.

In another preferred embodiment the cardiac arrhythmia is selected from paroxysmal, persistent, long lasting persistent or permanent (chronic) atrial fibrillation, typical atrial flutter, atypical atrial flutter, left atrial tachycardia, upper-loop tachycardia or other atrial macroreentrant tachycardias. Preferably, the cardiac arrhythmia is selected from focal atrial tachycardia or atrial premature beats. Even more preferably, the cardiac arrhythmia is selected from right, left or biatrial arrhythmias.

In another preferred embodiment the antagonist of TASK-1 K_{2P}3.1 is used in a subject, wherein the subject is healthy, or suffers from or is at risk of developing an atrial arrhythmia. It is preferred that
(i) the subject is suffering from or at risk of developing an atrial arrhythmia due to an underlying post ischemic contractile dysfunction, congestive heart failure, cardiogenic shock, septic shock, myocardial infarction, cardiomyopathy, dysfunction of heart valves, planned thoracotomy or ventricular disorder; and/or
(ii) the subject that is healthy, or suffers from or is at risk of developing an atrial arrhythmia carries one or more genetic mutations linked to development of atrial arrhythmias.
   More preferably, the subject exhibits increased risk scores for the development of atrial arrhythmias that can be calculated from clinical parameters as described by Kallenberger S M, Schmid C, Wiedmann F, Mereles D, Katus H A, et al. (2016) A Simple, Non-Invasive Score to Predict Paroxysmal Atrial Fibrillation. PLOS ONE 11(9): e0163621. https://doi.org/10.1371/journal.pone.0163621. Especially subjects with increased left atrial diameter (over 40 mm), age over 70 years, dilatation of the aortic rout diameter, increased velocity in the left atrium measured in the tissue by doppler ultrasound, sleep apnea, body mass index over 27 might show an increased risk for atrial arrhythmias. Even more preferably, the subject is suffering from right, left, or biatrial arrhythmias.

The first aspect of the invention comprises a second medical use directed to an antagonist of the Two-Pore Domain Potassium Channel (TASK-1) K_{2P}3.1 for use in the prevention and/or treatment of cardiac arrhythmia in a subject. The claim is a purpose-limited substance claim. Thus, the claimed antagonist of TASK-1 K_{2P}3.1 is suitable for a method of treatment for cardiac arrhythmia.

In a second aspect, the invention further relates to a nucleic acid molecule comprising a polynucleotide, wherein the polynucleotide comprises a nucleotide sequence selected from the group consisting of
(i) at least 10 consecutive nucleotides of the nucleotide sequence according to SEQ ID NO: 1, SEQ ID NO:4 and SEQ ID NO: 5, or variants thereof; or
(ii) the RNA encoded by (i); or
(iii) a complement of (i) or (ii).

In a preferred embodiment the nucleotide sequence comprises at least 10, more preferably at last 15, more preferably at least 20 consecutive nucleotides of the nucleotide sequence according to SEQ ID NO: 1 and most preferably the entire nucleotide sequence according to SEQ ID NO: 1. In a more preferred embodiment the nucleotide sequence comprises at least 10, more preferably at last 15, more preferably at least 20 consecutive nucleotides of the nucleotide sequence according to SEQ ID NO: 4 and most preferably the entire nucleotide sequence according to SEQ ID NO: 4. In an even more preferred embodiment the nucleotide sequence comprises at least 10, more preferably at last 15, more preferably at least 20 consecutive nucleotides of the nucleotide sequence according to SEQ ID NO: 5 and most preferably the entire nucleotide sequence according to SEQ ID NO: 5.

In another preferred embodiment that nucleotide sequence comprises at least 10 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 10 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 10 nucleotides of a variant of the nucleotide sequence according to SEQ ID NO: 5, wherein the variants have at least 85%, 90%, 95% or 99% sequence identity to SEQ ID NO: 5.

In another preferred embodiment that nucleotide sequence comprises at least 15 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 15 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 15 nucleotides of a variant of the nucleotide sequence according to SEQ ID NO: 5, wherein the variants have at least 85%, 90%, 95% or 99% sequence identity to SEQ ID NO: 5.

In another preferred embodiment that nucleotide sequence comprises at least 20 nucleotides of a variant of the nucleotide sequence according to SEQ ID NO: 1, wherein the variants have at least 85%, 90%, 95% or 99% sequence identity to SEQ ID NO: 1. In another preferred embodiment that nucleotide sequence comprises at least 20 nucleotides of a variant of the nucleotide sequence according to SEQ ID NO: 4, wherein the variants have at least 85%, 90%, 95% or 99% sequence identity to SEQ ID NO: 4. In another preferred embodiment that nucleotide sequence comprises at least 20 nucleotides of a variant of the nucleotide sequence according to SEQ ID NO: 5, wherein the variants have at least 85%, 90%, 95% or 99% sequence identity to SEQ ID NO: 5.

In another preferred embodiment that nucleotide sequence is a variant of the nucleotide sequence according to SEQ ID NO: 1, wherein the variants have at least 85%, 90%, 95% or 99% sequence identity to SEQ ID NO: 1. In another preferred embodiment that nucleotide sequence is a variant of the nucleotide sequence according to SEQ ID NO: 4, wherein the variants have at least 85%, 90%, 95% or 99% sequence identity to SEQ ID NO: 4. In another preferred embodiment that nucleotide sequence is a variant of the nucleotide sequence according to SEQ ID NO: 5, wherein the variants have at least 85%, 90%, 95% or 99% sequence identity to SEQ ID NO: 5.

In another preferred embodiment the nucleotide sequence is an RNA sequence encoded by the at least 10 nucleotides of the sequence according to SEQ ID NO: 1 or variants thereof. It is more preferred that the nucleotide sequence is an RNA sequence encoded by the at least 10 nucleotides of the sequence according to SEQ ID NO: 4 or variants thereof. It is even more preferred that the nucleotide sequence is an RNA sequence encoded by the at least 10 nucleotides of the sequence according to SEQ ID NO: 1 or variants thereof.

In another preferred embodiment the nucleotide sequence is an RNA sequence encoded by the nucleotides of the sequence according to SEQ ID NO: 1 or variants thereof. It is preferred that the nucleotide sequence is an RNA sequence encoded by the nucleotides of the sequence according to SEQ ID NO: 4 or variants thereof. It is even more preferred that the nucleotide sequence is an RNA sequence encoded by the nucleotides of the sequence according to SEQ ID NO: 5 or variants thereof.

In another preferred embodiment the nucleotide sequence comprises complements of the at least 10 consecutive nucleotides of the nucleotide sequence according to SEQ ID NO: 1, SEQ ID NO:4 and SEQ ID NO: 5. In another preferred embodiment the nucleotide sequence comprises complements of the variants of the at least 10 consecutive nucleotides of the nucleotide sequence according to SEQ ID NO: 1, SEQ ID NO:4 and SEQ ID NO: 5. In another preferred embodiment the nucleotide sequence comprises complements of the RNA encoded by the nucleotides of the sequence according to SEQ ID NO: 1, SEQ ID NO:4 and SEQ ID NO: 5. In another preferred embodiment the nucleotide sequence comprises complements of the RNA encoded by the nucleotides of the variants of the sequence according to SEQ ID NO: 1, SEQ ID NO:4 and SEQ ID NO: 5.

In a preferred embodiment nucleic acid comprises a siRNA wherein preferably the siRNA is comprised in a micro RNA precursor or derivatives thereof. More preferably, the siRNA is comprised in a pri-miRNA scaffold. Even more preferably the pri-miRNA scaffold is selected from the group consisting of pri-miR155 scaffold, pri-miR1 scaffold, pri-miR30 scaffold, pri-miR125b scaffold, or pri-miR150 scaffold. Even more preferably, the nucleic acid comprising the siRNA is selected from the group consisting of SEQ ID NO: 1, SEQ ID NO: 5, SEQ ID NO: 5, SEQ ID NO: 16, SEQ ID NO: 17, SEQ ID NO: 18, SEQ ID NO: 19 or variants thereof. Most preferably, the siRNA is SEQ ID NO: 1, SEQ ID NO: 4, SEQ ID NO: 5 or variants thereof. It is preferred that the siRNA sequences according to the SEQ IDs above further comprise ITR sequences.

It is noted that the nucleic according to the second aspect of the invention may comprise additional elements or preferred embodiments as outlined in detail in relation to the nucleic used in the first aspect of the invention.

In a third aspect, the invention relates to a vector comprising the inhibitory nucleic acid of the first and second aspect of the invention. In a preferred embodiment the vector comprising the inhibitory siRNA is selected from the group consisting of plasmid vectors, cosmid vectors, and viral vectors. More preferably, the vector is a viral vector and is even more preferably selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector or phage vector. Even more preferably, the vector is an AAV and is selected from the group consisting of AAV1, AAV2, AAV3, AAV4, AAV5, AAV6, AAV7, AAV8, AAV9, AAV10, AAVrh10, AAV11, and AAV12 or variants thereof with a tropism to heart tissue. Most preferably the vector is an AAV2, AAV6 and AAV9 vector. Most, most preferably the vector is an AAV9 and/or variants of AAV9, showing an increased tropism to heart tissue as described above.

In another embodiment it is preferred that the nucleic acid comprised in the vector is operably linked to an expression control sequence. Preferably the expression control sequence is a heart tissue specific promoter. Even more preferably the heart-tissue specific promoter is selected from the group consisting of Cardiac Actin Enhancer/Elongation Factor 1 promoter Cytomegali-virus enhancer/Myosin light chain ventricle 2 promoter, troponin, Atrial Natriuretic Peptide or Slow Myosin Heavy Chain 3 G. Even more preferably, the heart-tissue specific promoter is troponin.

It is noted that the vector according to the third aspect of the invention may comprise additional elements or preferred embodiments as outlined in detail in relation to the vector used in the first aspect of the invention.

In a fourth aspect the invention relates to a cell comprising the nucleic acid according to the second aspect of the invention and/or the vector of the third aspect of the invention. It is preferred that cells comprise any of the nucleic acids of the second aspects. It is further preferred that the cell is a reprogrammed cell with a cardiac phenotype. Even more preferably such a cell is an induced pluripotent stem cell. It is also preferred that the cell is a heart cell, more preferably a human heart cell. Even more preferably the heart cell is an atrial heart cell.

### EXAMPLES

### Example 1

Molecular Biology and Plasmid Generation

For generation of pSSV9-TNT-miR/si-p/hTASK-1-x-IRES2-eGFP and pSSV9-CMV/MLC-miR/si-p/hTASK-1-x-IRES2-eGFP the IRES2 element was excised from pIRES2-DsRed-Express (Clontech Laboratories Inc., Mountain View, Calif., USA) via Ncol/BamHI and subcloned in pSSV-CMV/MLC260-eGFP and pSSV-TnT-eGFP. CDNAs encoding for pri-miR155 embedded TASK-1 siRNAs 1-3 were amplified from custom synthesized single stand oligonucleotides (Sigma Aldrich, Steinheim, Germany) via PCR using primers, carrying SacII and BamHI restriction sites. This restriction sites were used for directional cloning in the abovementioned IRES2-carrying pSSV9 plamids. The pSSV-TnT-eGFP construct was used for production of control AAV9-Tnt-eGFP vectors.

Adeno-Associated Virus Generation

High titer vectors were produced, using a double transfection approach of HEK 293T cells in cell stacks (Corning, Munich, Germany) as described before (Jungmann A et al. 2017, Hum Gene Ther Methods https://www.ncbi.nlm.nih.gov/pubmed/28934862doi: 10.1089/hum.2017.192). For production of AAV9-siTASK-1-eGFP pDP9rs, providing the AAV-9 cap sequence was co-transfected with pSSV9-TNT-miR/si-p/hTASK-1-x-IRES2-eGFP or pSSV9-CMV/MLC-miR/si-p/hTASK-1-x-IRES2-eGFPinto HEK293T. For in vitro studies on neonatal rat cardiomyocytespDP6rs plasmid was used, yielding AAV serotype 6 vectors.

After 48 h cells were harvested, lysis was performed by 4-8 freeze-thawing cyclesin the presence of protease inhibitors (protease inhibitor mix G, SERVA Electrophoresis GmbH, Heidelberg, Germany). Vectors were purified by filtration (0.2 μm) and iodixanol step gradient ultracentrifugation. Quantification was performed using SYBR-green based real time qPCR as reported earlier (Jungmann A et al. 2017, Hum Gene Ther Methods https://www.ncbi.nlm.nih.gov/pubmed/28934862doi: 10.1089/hum.2017.192).

### Isolation and Cultivation of Neonatal Rat Cardiomyocytes

Neonatal rat myocardial cells were dispersed from the ventricles of 1-3-day-old Sprague-Dawley rats by digestion with collagenase I (Worthington Biochemical Corporation, Lakewood, N.J., USA) and pancreatin (GIBCO, Thermo Fisher Scientific, Waltham, Mass., USA) at 37° C. The cell suspensions were separated on a discontinuous percoll gradient to obtain myocardial cell cultures with >99% cardiomyocytes. The cells were plated in T75 culture flasksin 4:1 Dulbecco's modified Eagle's medium (DMEM)/medium 199 (GIBCO, Thermo Fisher Scientific), supplemented with 10% fetal calf serum, 5% horse serum and penicillin streptomycin mix. After 24 h, cardiomyocytes were infected with AAV6. Infection was controlled by visualization of the eGFP reporter using epifluorescence microscopy. Cells were harvested 48-72 h post infection using RIPA buffer as described before (Schmidt et al. 2017, Eur Heart J 38:1764-1774).

Proteinbiochemistry and Immunoblotting

Protein concentration of cell lysates was determined using the bicinchoninic acid (BCA) protein assay (Thermo Fisher Scientific) according to the manufacturer's protocol. Protein samples were diluted in Laemmli buffer containing 5% beta-mercaptoethanol and boiled for 5 min. Immunodetection of TASK-1 protein was performed after sodium dodecyl sulfate (SDS) gel electrophoresis and wet transfer to nitrocellulose membranes as described (Schmidt et al. 2017, Prog Biophys Mol Biol S0079-6107(17)30028-7). Membranes were developed by sequential exposure to a blocking solution containing 3% bovine serum albumin and 5% dry milk, primary antibodies directed against TASK-1 (1:400; APC-024, Alomone Labs, Jerusalem, Israel) and appropriate horseradish peroxidase (HRP)-conjugated secondary antibodies (1:3000; NA934V, GE Healthcare, Munich, Germany). Signals were developed using the enhanced chemiluminescence assay (ECL Western Blotting Reagents, GE Healthcare, Buckinghamshire, UK) and quantified with ImageJ 1.41 Software (National Institutes of Health, Bethesda, Md., USA). Protein content was normalized to glyceraldehyde 3-phosphate dehydrogenase (GAPDH) using anti-GAPDH primary antibodies (1:10.000; G8140-11; US Biological, Swampscott, Mass., USA) and corresponding secondary antibodies (1:3000, sc-2005, Santa-Cruz Biotechnology, Heidelberg, Germany) for quantification of optical density. The pSSV9-TNT-miR-siTASK-1-IRES2-eGFP vector (hereafter pSSV9-siTASK-1-eGFP) contains TASK-1 siRNA, embedded in a pri-miR155 scaffold and the cDNA of a reporter protein (eGFP) separated by an internal ribosome entry site (IRES2). Expression of these cDNAs is controlled by the cardiac troponin promotor which allows for cardiomyocyte specific expression. The plasmid carries two inverted terminal repeat sequences (ITR). Furthermore an ampicillin resistance gene allows for amplification of the plasmids in E. coli. Cardiotrophic AAV9 vectors containing single strand DNA were used for large animal experiments, while AAV6 vectors containing single strand DNA were used for in vitro tests in neonatal rat cardiomyocytes.

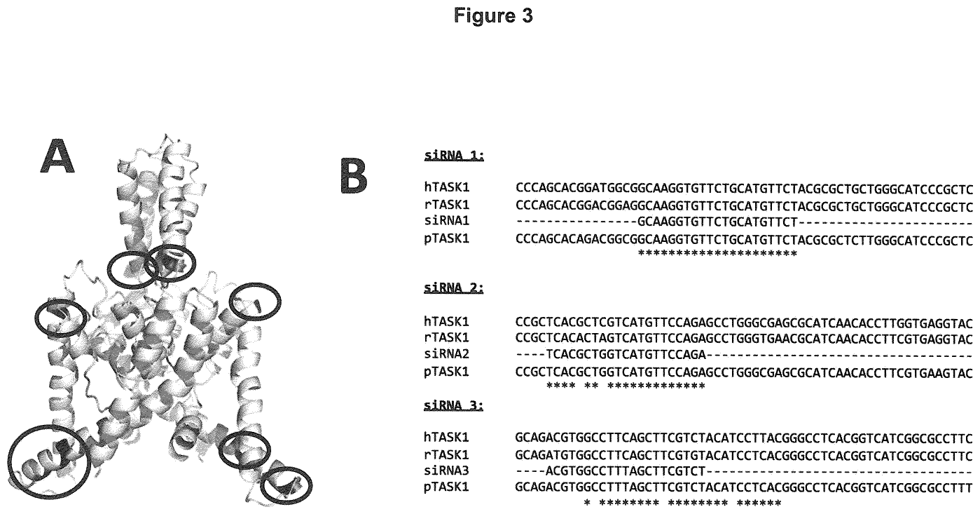

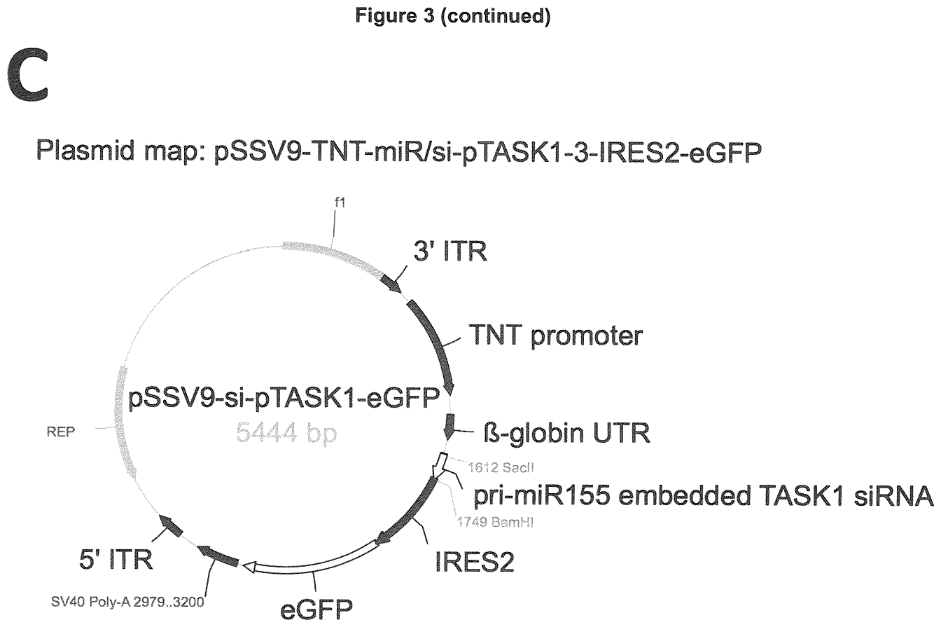

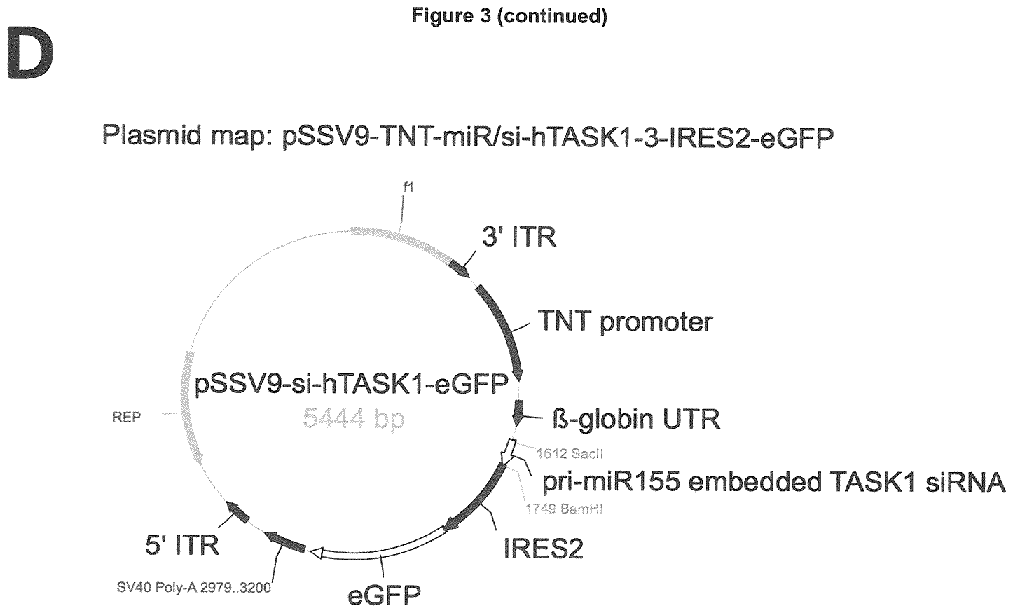

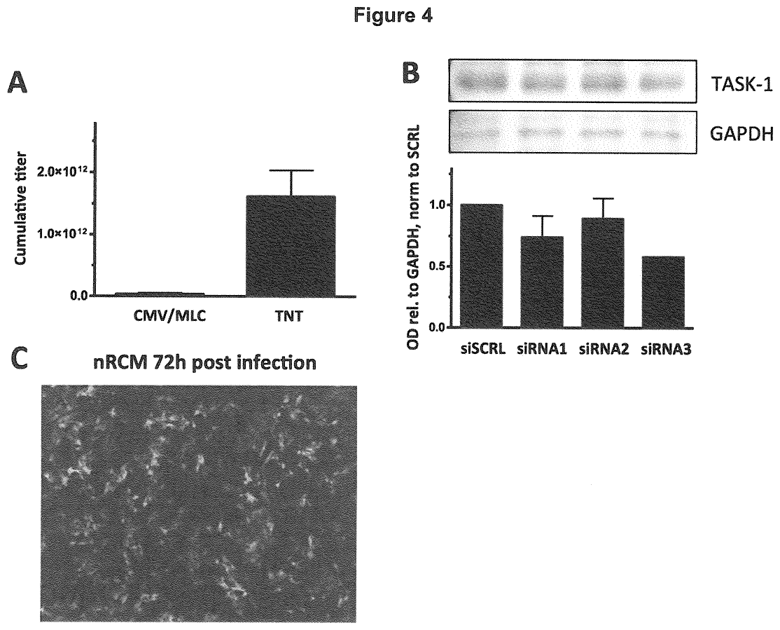

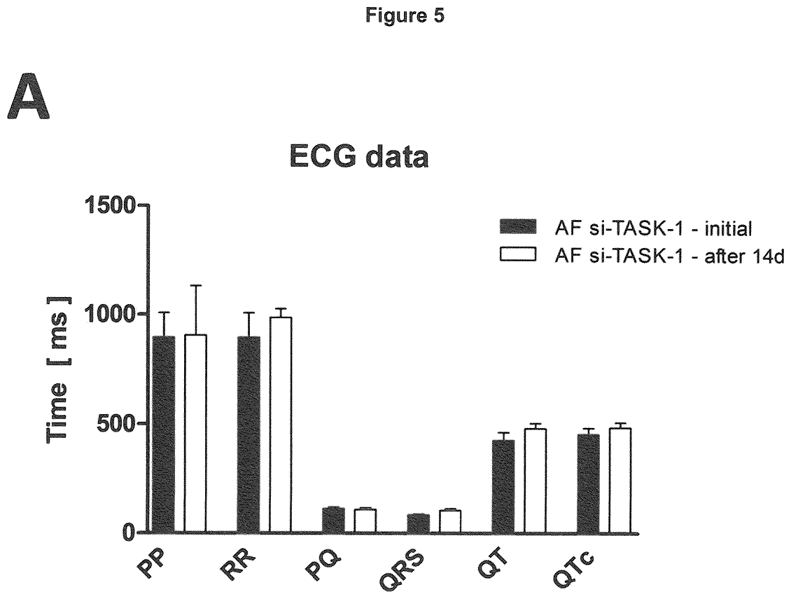

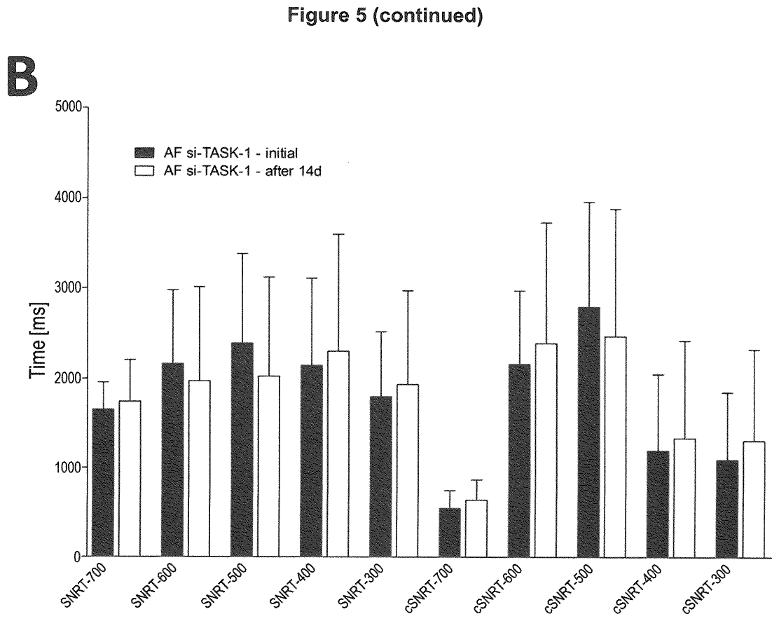

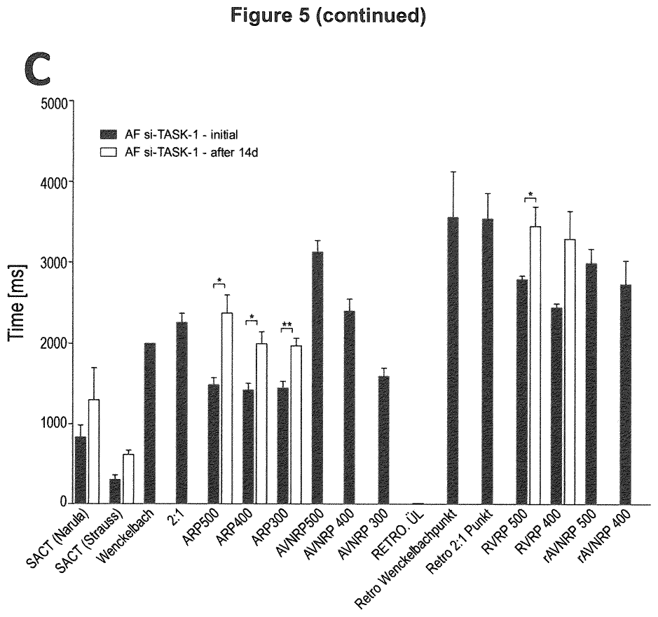

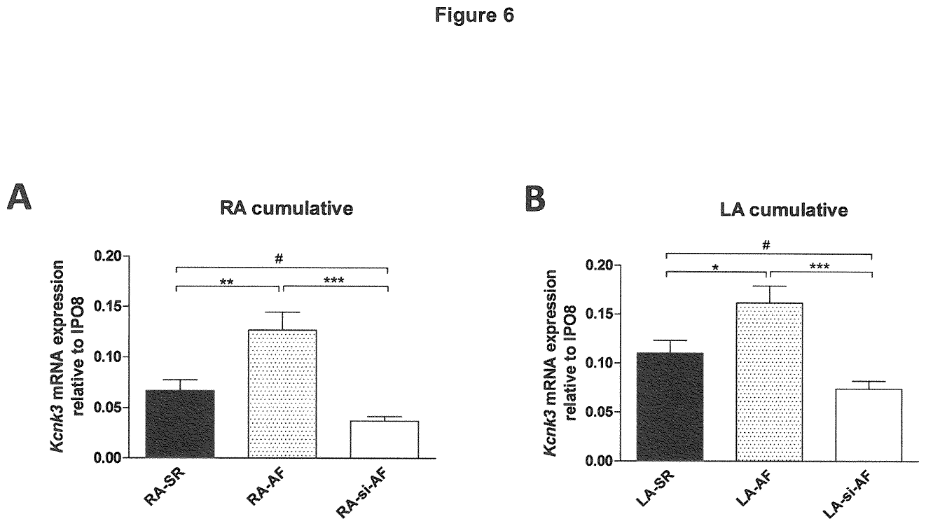

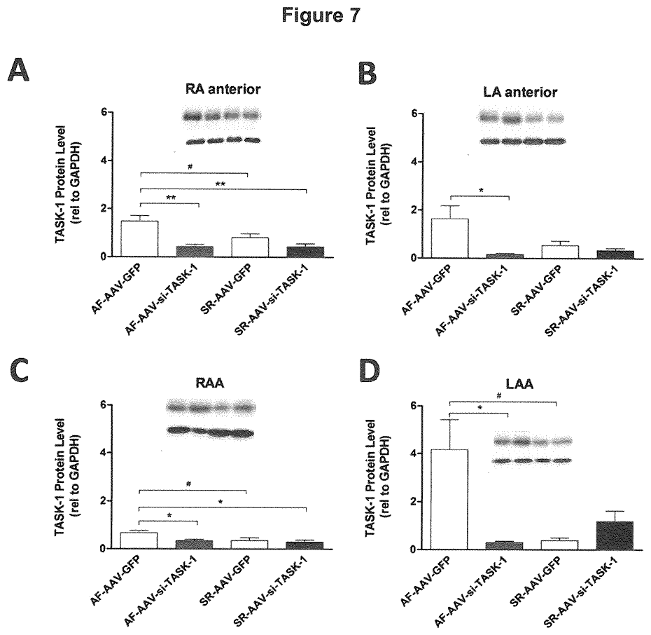

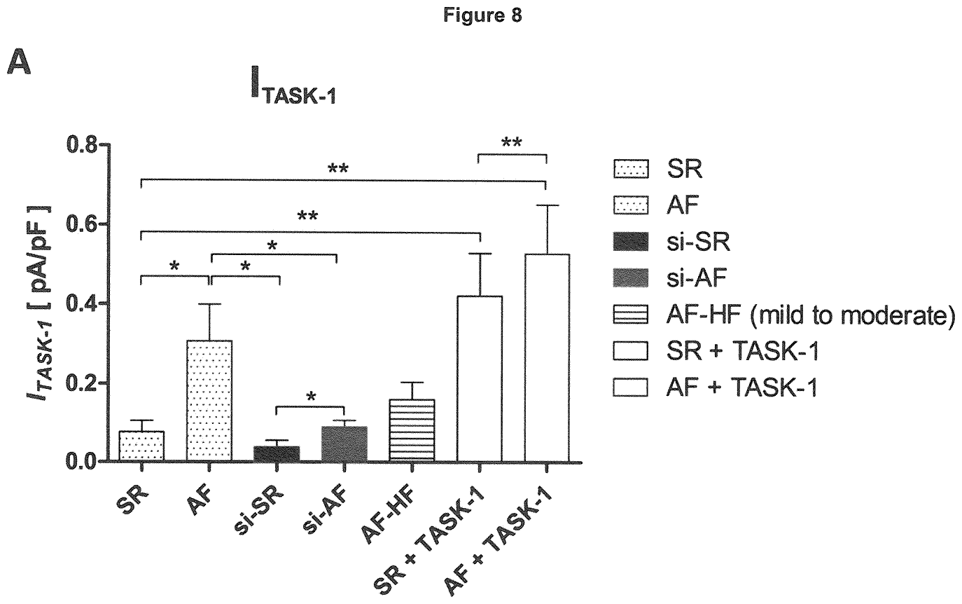

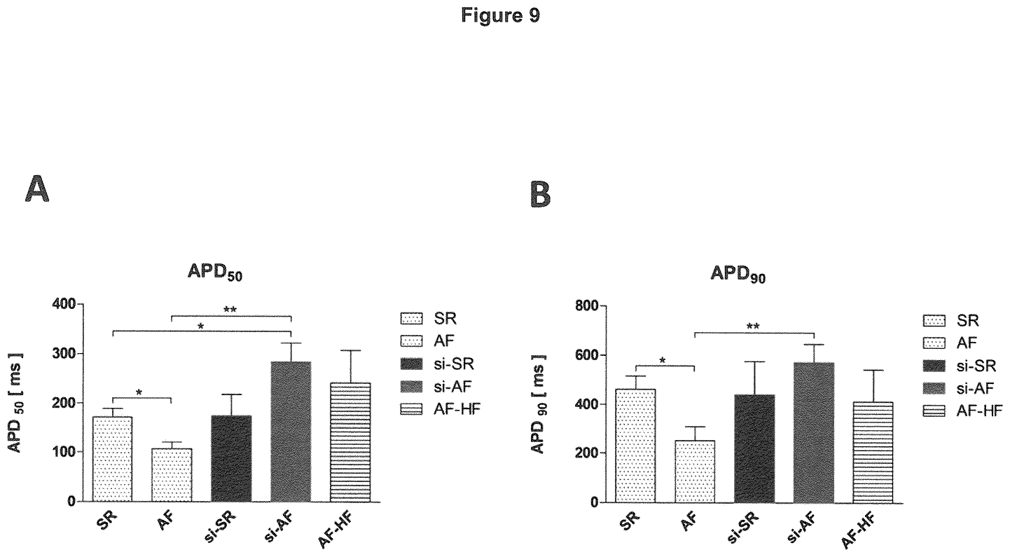

Two cardiomyocyte specific promoters, a CMV-enhanced 260-bp myosin light chain (MLC260) promoter and a troponin T (TNT, i.e. hTNNT2v1) were tested to control the miR-siTASK-1-IRES2-eGFP cassette. Surprisingly, AAV9 production using pSSV9-CMV/MLC260-miR-siTASK-1-IRES2-eGFP yielded very low titer when compared to pSSV9-TNT-miR-siTASK-1-IRES2-eGFP (FIG. 3A), therefore all constructs used in further studies were under control of the cardiac specific TNT promoter.

Three siRNA sequences, directed against the porcine orthologue of TASK-1 were subjected to in vitro efficacy tests in cultured neonatal rat cardiomyocytes. As infection with AAV6 particles carrying siRNA sequence number 3 yielded best results for downregulation of TASK-1 protein levels in neonatal rat cardiomyocytes (FIG. 3B), pSSV9-TNT-miR-siTASK-1-3-IRES2-eGFP was chosen for in vivo studies. FIG. 3c depicts eGFP fluorescence signal of neonatal rat cardiomyocytes 72 h post infection with AAV6-siTASK-1-eGFP.

### Example 2

Porcine Atrial Fibrillation Model

Animal experiments have been carried out in accordance with the Guide for the Care and Use of Laboratory Animals as adopted and promulgated by the U.S. National Institutes of Health (NIH publication No. 86-23, revised 1985) and with EU Directive 2010/63/EU, and the current version of the German Law on the Protection of Animals was followed. Approval for experiments involving pigs was granted by the local Animal Welfare Committee in Heidelberg (Germany, reference number G-296/14). Induction of atrial fibrillation in domestic pigs was carried out by rapid atrial burst pacing via an implanted cardiac pacemaker (St. Jude Medical, St. Paul, Minn., USA). After 20 seconds of high-rate atrial pacing at 40 Hz burst pacing was paused to evaluate if AF persisted. Whenever the pigs returned to sinus rhythm for >15s, episodes of burst pacing were started again. Domestic swine of both gender (20-50 kg), were randomized to either AF induction by activation of atrial burst pacing or SR. Anesthesia was performed using azaperone, midazolam and propofol or during thoracotomy isoflurane. To prevent from tachycardia induced heart failure, prior to AF induction AV-node ablation was performed under electrocardiographic and fluoroscopic guidance. AAV9 preparations were applied by direct injection into porcine atria (31.33±0.57 injections per atrium, at a titer of 3×10¹²vgc per animal) after thoracotomy. On day 0 and prior to final operation on day 14 pigs were subjected to clinical examination, 12-channels ecgs, pacemaker interrogation, detailed echocardiography and EP-studies. During the 14 day follow up period, clinical examinations and 4 channel ECGs were performed on a daily basis.

Electrophysiological Examination

EP studies were performed in all animals at baseline condition (i.e. on day 0 prior to pacemaker implantation and thoracotomy) and on day 14. Prior to or during EP-studies no volatile anesthetics were used to avoid interaction pharmacological with cardiac two-pore-domain potassium channels. If persistent AF episodes required electrical cardioversion, EP studies were paused for at least 30 min afterwards. After cannulation of the jugular vein, quadripolar catheters were placed under fluoroscopic guidance at the junction of the superior vena cava to the right atrium and in the right ventricular apex. A UHS 20 stimulus generator (Biotronik, Berlin, Germany) was used for intracardiac stimulation and the EP Lab duo system (Bard Electrophysiology Division, Lowell, Mass., USA) was used for recording, analyzing and storing electrocardiograms. Parameters were measured according to clinical conventions.

Cardiomyocyte Isolation

Immediately after excision, atrial tissue samples were dissected into small pieces, and rinsed 3 times in Ca²⁺-free Tyrode's solution (in mM: NaCl 100, KCl 10, KH₂PO₄1.2, MgSO₄ 5, taurine 50, 3-(N-morpholino) propanesulfonic acid (MOPS) 5 and glucose 20, pH 7.0 with NaOH) supplemented with 2,3-butanedione monoxime (BDM, 30 mM; Sigma-Aldrich, St. Louis, Mo., USA). The solutions were oxygenated with 100% O₂ at 37° C. After digestion with collagenase type I (288 U/ml, Worthington) and protease type XXIV (5 mg/ml Sigma-Aldrich) for 15 minutes, Ca²⁺ concentration was increased to 0.2 mM. Following agitation in protease-free solution for another 35 min, rod-shaped single cardioymyocytes could be harvested. For storage until usage in patch-clamp experiments, suspension was centrifuged, and cells were resuspended in storage medium (in mM: KCl 20, KH₂PO₄ 10, glucose 10, K glutamate 70, β-hydroxybutyrate 10, taurine 10, ethylene glycol tetraacetic acid (EGTA) 10 and 1% of albumin).

Cellular Electrophysiology

Patch clamp glass pipettes, pulled from borosilicate glass (1B120E-4; World Precision Instruments, Berlin, Germany) had tip resistances ranging from 3 to 4 MΩ after back-filling with patch clamp internal solution (in mM: KCl 60, K glutamate 65, K₂ATP 3, Na₂GTP 0.2, MgCl₂ 2, EGTA 5, 4-(2-hydroxyethyl)piperazine-1-ethanesulfonic acid (HEPES) 5, (pH7.2 with KOH). All experiments were carried out at room temperature under constantly superfusion with extracellular solution containing (in mM): NaCl 140, KCl 5.4, MgCl₂1, CaCl₂ 1, NaH₂PO₄ 0.33, HEPES 5, glucose 10 (pH 7.4 with NaOH). Data were not corrected for liquid junction potentials and no leak subtraction was performed. Membrane currents were evoked by application of voltage steps between −80 and +80 mV in 10 mV-increments (duration, 300 ms; holding potential, −50 mV). Patch clamp internal solution for current clamp recordings was composed as follows (in mM): K gluconate 134, NaCl 6, MgCl₂ 1.2, MgATP 1, HEPES 10 (pH adjusted to 7.2 with KOH) and extracellular Tyrode's solution consisted of NaCl 137, KCl 5.4, CaCl₂2, MgSO₄ 1, glucose 10 and HEPES 10 (pH 7.3 with NaOH).

Real-Time qPCR

For isolation of RNA from flash frozen tissue samples, TRIzol-Reagent (Thermo Fisher Scientific) was used according to the manufacturer's instructions. After quantification by spectrophotometry (NanodropND 1000, Thermo Fisher) single-stranded cDNA was generated, as described earlier (Schmidt et al. 2015, Circulation 132:82-92) with the Maxima First Strand cDNA Synthesis Kit (Thermo Fisher Scientific), using 3 μg of total RNA per 20 μl reaction. Quantitative real-time PCR (qPCR) was carried out as reported (Schmidt et al. 2017, Eur Heart J 38:1764-1774). In short, 10 μl, consisting of 0.5 μl cDNA, 5 μl TaqMan Fast Universal Master Mix (Thermo Fisher Scientific), and 0.5 μl 6-carboxyfluorescein (FAM)-labeled TaqMan probes and primers (KCNK3, HS00605529 ml, TaqMan Gene Expression Assays; Thermo Fisher Scientific) per reaction were analyzed, using the StepOnePlus (Applied Biosystems, Foster City, Calif., USA) PCR system. The beta actin (ACTB, SS03376081_ul, TaqMan Gene Expression Assays; Thermo Fisher Scientific) housekeeping gene was used for normalization. All RT-qPCR reactions were performed in triplicates and control experiments in the absence of cDNA were included. Means of triplicates were used for the 2^{-ΔCt} calculation, where 2^{-Δct} corresponds to the ratio of mRNA expression versus ACTB.

Histology

For histological analysis, atrial tissue samples were taken from the left and right atrial appendages. Sample sites were similar among all study pigs. Atrial preparations were fixed in Tissue-Tek Compound (Sakura Finetek, Staufen, Germany) and frozen in fluid nitrogen. Frozen sections were cut to 10 μm thickness and stored at −80° C. Sections were thawed prior to immunostaining, fixed in cold acetone, and dried at room temperature. After rinsing with phosphate buffered saline (PBS), the sections were blocked in 1×PBS supplemented with 0.5% triton, 1% BSA, and 10% goat serum. eGFP immunostaining (green fluorescence) was detected using monoclonal mouse anti GFP antibodies (1:1000; MA5-1526565; Thermo) and Alexa Fluor 488-conjugated secondary antibodies (1:1000; A-11055; Thermo Fisher Scientific).

Results of KCNK3 Based Gene-Therapy in Pigs

Electrophysiological Examinations

The atrial refractory period was significantly prolonged after 14 days of anti-TASK-1 AAV treatment. Furthermore, the right ventricular refractory period was also significantly prolonged at 500 ms compared to pigs with only AF over 14 days. In pigs with AF, anti-TASK-1 AAVs significantly reduced AF inducibility compared to untreated AF pigs (s. FIG. 5).

mRNA and Protein Analysis

At the molecular level, AAV treatment resulted in down regulation of TASK-1 at mRNA and protein levels in the right and left atrium. The highest TASK-1 expression levels were found in the right and left atrial appendage. TASK-1 showed a restricted expression to the right and left atrium (see FIG. 6 and FIG. 7).

Electrophysiological Investigations of Pig Cardiomyocytes

Electrophysiological recordings from isolated pig cardiomyocytes showed significantly reduced TASK-1 currents after anti-TASK-1 AAV treatment. TASK-1 currents were significantly increased in cardiomyocytes of pigs with TASK-1 overexpression after AAV gene transfer (s. FIG. 8). AF was associated with shortening of atrial APD in pigs without gene therapy. By contrast, anti-TASK-1 gene therapy in AF pigs prolonged the atrial APD significant (see FIG. 9).

1. A method for preventing and/or treating cardiac arrhythmia, comprising administering to a subject in need thereof an antagonist of the Two-Pore Domain Potassium Channel (TASK-1) K_{2P}3.1.

2. The method of claim 1, wherein the antagonist:
(i) is an inhibitor of translation of TASK-1 encoding mRNA;
(ii) is an inhibitor of transcription of TASK-1 encoding mRNA or the processing thereof;
(iii) is an inhibitor of maturation, post-translational modification, trafficking, recycling, or degradation or activity of TASK-1; or
(iv) is an inhibitor of the function of TASK-1.

3. The method of claim 2, wherein the inhibitor of translation of TASK-1 encoding mRNA is selected from the group consisting of nucleic acids, e.g. s siRNA or shRNA, miRNA or lncRNA, microRNA-sponges, anti-miRNA oligonucleotides, chemically modified miRNA mimics, pre-miRNA, pri-miRNA, anti-pre-miRNA oligonucleotides, anti-pri-miRNA oligonucleotides, or derivatives thereof.

4. The method of claim 2, wherein the inhibitor of transcription of TASK-1 encoding mRNA is selected from the group consisting of nucleic acids, proteins or compositions thereof, modifying methylation of genomic DNA, folding of genomic DNA and histone phosphorylation or the accessibility of translation initiators, enhancers or genomic DNA encoding for TASK-1 mRNA.

5. The method of claim 3, wherein the nucleic acid molecule is comprised in a pri-miRNA scaffold.

6. The method of claim 3, wherein the nucleic acid molecule comprises a polynucleotide, wherein the polynucleotide comprises a nucleotide sequence selected from the group consisting of
(i) at least 10 consecutive nucleotides of the nucleotide sequence according to SEQ ID NO: 1, SEQ ID NO: 4, SEQ ID NO: 5, SEQ ID NO: 16, SEQ ID NO: 17, SEQ ID NO: 18, SEQ ID NO: 19 or variants thereof; or
(ii) the RNA encoded by (i);
(iii) a complement of (i) or (ii).

7. The method of claim 2, wherein the inhibitor of maturation, post-translational modification, trafficking, recycling, or degradation or activity of TASK-1 protein is selected from the group consisting of the N-glycosylation inhibitor tunicamycin, the 14-3-3 inhibitor 2-(2,3-Dihydro-1,5-dimethyl-3-oxo-2-phenyl-1H-pyrazol-4-yl)-2,3-dihydro-1,3-dioxo-1H-isoindole-5-carboxylicacid, siRNA downregulating P11 or β-COP.

8. The method of claim 2, wherein the inhibitor of the function of TASK-1 is selected from the group consisting of a ligand specifically binding to TASK-1, a nucleic acid encoding such a ligand, a protein or compound increasing phosphorylation of TASK-1.

9. The method of claim 8, wherein:
(a) the ligand is selected from the group consisting of antibodies, antigen-binding fragments of antibodies, antibody-like proteins, 2-(Butane-1-sulfonylamino)-N—[(R)-1-(6-methoxy-pyridin-3-yl)-propyl]-benzamide (A293), N-[(2,4-difluorophenyl)-methyl]-2-[2-[[[2-(4-methoxyphenyl)-acetyl]-amino]-methyl]-phenyl]-benzamide (A1899), 2-Methoxy-N-[3-[(3-methylbenzoyl)-amino]-phenyl]-benzamide (ML365); or
(b) the protein or compound modulating phosphorylation of TASK-1 is selected from the group consisting of endothelin-1, platelet activating factor, PKCε activator εRACK, serotonin, thyrotropin releasing hormone (TRH), acetylcholine, angiotensin II or α-adrenergic agonist methoxamine.

10. The method of claim 4, wherein the nucleic acid is comprised in a vector.

11. The method of claim 10, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector or phage vector.

12. The method of claim 11, wherein the AAV vector is selected from the group consisting of AAV1, AAV2, AAV3, AAV4, AAV5, AAV6, AAV7, AAV8, AAV9, AAV10, AAVrh10, AAV11, and AAV12 and variants thereof with a tropism to heart tissue.

13. The method of claim 10, wherein the nucleic acid is operably linked to an expression control sequence.

14. The method of claim 13, wherein the heart-tissue specific promoter is selected from the group consisting of Cardiac Actin Enhancer/Elongation Factor 1 promoter Cytomegali-virus enhancer/Myosin light chain ventricle 2 promoter, troponin, Atrial Natriuretic Peptide or Slow Myosin Heavy Chain 3 Gene.

15. The method of claim 1, wherein the antagonist is administered peroral, inhalative, by intravenous, intramucosal, intraarterial, intramusculuar, intracardiac, intraatrial or intracoronal injection.

16. The method of claim 10, wherein the viral vector is administered in a dosage of 1×10¹¹-1×10¹⁴ viral particles per dose.

17. The method of claim 1, wherein the cardiac arrhythmia is selected from paroxysmal, persistent, long lasting persistent or permanent (chronic) atrial fibrillation.

18. The method of claim 1, wherein the cardiac arrhythmia is selected from the group consisting of typical atrial flutter, atypical atrial flutter, left atrial tachycardia, upper-loop tachycardia, right atrial arrhythmias, left atrial arrhythmias, biatrial arrhythmias or other atrial macroreentrant tachycardias.

19. The method of claim 1, wherein the subject is healthy, or suffers from or is at risk of developing an atrial arrhythmia.

20. The method of claim 1, wherein the subject exhibits increased risk scores for the development of atrial arrhythmias.

21. A nucleic acid molecule comprising a polynucleotide, wherein the polynucleotide comprises a nucleotide sequence selected from the group consisting of
(i) at least 10 consecutive nucleotides of the nucleotide sequence according to SEQ ID NO: 1, SEQ ID NO: 4 and SEQ ID NO: 5, or variants thereof; or
(ii) the RNA encoded by (i);
(iii) a complement of (i) or (ii).

22. The nucleic acid according to claim 21 wherein the RNA encoded by (ii) or complements thereof is siRNA and is comprised in a pri-miRNA scaffold.

23. The nucleic acid according to claim 21 (ii), wherein the siRNA is selected from the group consisting of SEQ ID NO: 1, SEQ ID NO: 4, SEQ ID NO: 5, SEQ.

24. A vector comprising the nucleic acid according to claim 21, wherein the vector is preferably selected from the group consisting of plasmid vectors, cosmid vectors, and viral vectors.

25. The vector according to claim 24, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector or phage vector.

26. A vector according to claim 25, wherein the AAV vector is selected from the group consisting of AAV1, AAV2, AAV3, AAV4, AAV5, AAV6, AAV7, AAV8, AAV9, AAV10, AAVrh10 AAV11, and AAV12 and variants thereof with a tropism to heart tissue, preferably AAV2, AAV6, AAV9 and AAV9 variants.

27. The vector according to claim 25, wherein the nucleic acid is operably linked to an expression control sequence, preferably a heart tissue specific promoter.

28. The vector according to claim 27, wherein the heart tissue specific promoter is selected from the group consisting of Cardiac Actin Enhancer/Elongation Factor 1 promoter Cytomegali-virus enhancer/Myosin light chain ventricle 2 promoter, troponin, Atrial Natriuretic Peptide or Slow Myosin Heavy Chain 3 Gene, preferably troponin.

29. A cell comprising the nucleic acid according to 21.

30. The cell according to claim 29, wherein the cell is
(i) a reprogrammed cell with a cardiac phenotype; or
(ii) a heart cell;

31. The cell according to claim 30, wherein the heart cell is an atrial heart cell.


SEQUENCE LISTING (28 sequences)

SEQ ID NO: 1 - DNA, 21 bp, Artificial Sequence
  modifed sequence
  si-p/hTASK-1-1 (1)..(21): DNA sequence corresponding to the siRNA sequence 1 directed against the porcine and the human ortholog of TASK-1
gcaaggtgttctgcatgttct

SEQ ID NO: 2 - DNA, 21 bp, Artificial Sequence
  modified sequence
  si-pTASK-1-2 (1)..(21): DNA sequence corresponding to the siRNA sequence 2 directed against the porcine ortholog of TASK-1
tcacgctggtcatgttccaga

SEQ ID NO: 3 - DNA, 21 bp, Artificial Sequence
  Modified sequence
  si-pTASK-1-3 (1)..(21): DNA sequence corresponding to the siRNA sequence 3 directed against the porcine ortholog of TASK-1
acgtggcctttagcttcgtct

SEQ ID NO: 4 - DNA, 21 bp, Artificial Sequence
  modified sequence
  si-hTASK-1-2 (1)..(21): DNA sequence corresponding to the siRNA sequence 2 directed against the human ortholog of TASK-1
tcacgctagtcatgttccaga

SEQ ID NO: 5 - DNA, 21 bp, Artificial Sequence
  Modified sequence
  si-hTASK-1-3 (1)..(21): DNA sequence corresponding to the siRNA sequence 3 directed against the human ortholog of TASK-1
acgtggccttcagcttcgtct

SEQ ID NO: 6 - DNA, 5444 bp, Artificial Sequence
  Modified sequence
  pSSV9-TNT-miR/si-p/hTASK-1-1-IRES2-eGFP (1)..(5444): Plasmid carrying the ITR-flanked construct of TASK-1 siRNA sequence 1 (directed against the porcine and the human TASK-1 ortholog) coupled to an eGFP-Reporter via IRES2 under control of the cardiomyocyte specific Troponin promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagaacatgcagaacaccttgcgttttggccactgactgacgcaaggtgctgcatgttctcaggacacaaggcctgttactagcactcacatggaacaaatggcggatccctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 7 - DNA, 5444 bp, Artificial Sequence
  Modified sequence
  pSSV9-TNT-miR/si-pTASK-1-2-IRES2-eGFP (1)..(5444): Plasmid carrying the ITR-flanked construct of TASK-1 siRNA sequence 2 (directed against the porcine TASK-1 ortholog) coupled to an eGFP-Reporter via IRES2 under control of the cardiomyocyte specific Troponin promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgtctggaacatgaccagcgtgagttttggccactgactgactcacgctgcatgttccagacaggacacaaggcctgttactagcactcacatggaacaaatggcggatccctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 8 - DNA, 5444 bp, Artificial Sequence
  Modified sequence
  pSSV9-TNT-miR/si-pTASK-1-3-IRES2-eGFP (1)..(5444): Plasmid carrying the ITR-flanked construct of TASK-1 siRNA sequence 3 (directed against the porcine TASK-1 ortholog) coupled to an eGFP-Reporter via IRES2 under control of the cardiomyocyte specific Troponin promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagacgaagctaaaggccacgtgttttggccactgactgacacgtggcctagcttcgtctcaggacacaaggcctgttactagcactcacatggaacaaatggcggatccctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - DNA, 5444 bp, Artificial Sequence
  Modified sequence
  pSSV9-TNT-miR/si-hTASK-1-2-IRES2-eGFP (1)..(5444): Plasmid carrying the ITR-flanked construct of TASK-1 siRNA sequence 2 (directed against the human TASK-1 ortholog) coupled to an eGFP-Reporter via IRES2 under control of the cardiomyocyte specific Troponin promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgtctggaacatgacgagcgtgagttttggccactgactgactcacgctccatgttccagacaggacacaaggcctgttactagcactcacatggaacaaatggcggatccctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 5444 bp, Artificial Sequence
  Modified sequence
  pSSV9-TNT-miR/si-hTASK-1-3-IRES2-eGFP (1)..(5444): Plasmid carrying the ITR-flanked construct of TASK-1 siRNA sequence 3 (directed against the human TASK-1 ortholog) coupled to an eGFP-Reporter via IRES2 under control of the cardiomyocyte specific Troponin promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagacgaagctgaaggccacgtgttttggccactgactgacacgtggcctagcttcgtctcaggacacaaggcctgttactagcactcacatggaacaaatggcggatccctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 5859 bp, Artificial Sequence
  Modified sequence
  pSSV9-CMV/MLC-miR/si-p/hTASK-1-1-IRES2-eGFP (1)..(5859): Plasmid carrying the ITR-flanked construct of TASK-1 siRNA sequence 1 (directed against the porcine and the human TASK-1 ortholog) coupled to an eGFP-Reporter via IRES2 under control of the cardiomyocyte specific CMV-enhanced MLC260 promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagaacatgcagaacaccttgcgttttggccactgactgacgcaaggtgctgcatgttctcaggacacaaggcctgttactagcactcacatggaacaaatggcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - DNA, 5859 bp, Artificial Sequence
  Modified sequence
  pSSV9-CMV/MLC-miR/si-pTASK-1-2-IRES2-eGFP (1)..(5859): Plasmid carrying the ITR-flanked construct of TASK-1 siRNA sequence 2 (directed against the porcine TASK-1 ortholog) coupled to an eGFP-Reporter via IRES-2 under control of the cardiomyocyte specific CMV-enhanced MLC260 promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgtctggaacatgaccagcgtgagttttggccactgactgactcacgctgcatgttccagacaggacacaaggcctgttactagcactcacatggaacaaatggcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - DNA, 5859 bp, Artificial Sequence
  Modified sequence
  pSSV9-CMV/MLC-miR/si-pTASK-1-3-IRES2-eGFP (1)..(5859): Plasmid carrying the ITR-flanked construct of TASK-1 siRNA sequence 3 (directed against the porcine TASK-1 ortholog) coupled to an eGFP-Reporter via IRES2 under control of the cardiomyocyte specific CMV enhanced MLC260 promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagacgaagctaaaggccacgtgttttggccactgactgacacgtggcctagcttcgtctcaggacacaaggcctgttactagcactcacatggaacaaatggcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 5859 bp, Artificial Sequence
  Modified sequence
  pSSV9-CMV/MLC-miR/si-hTASK-1-2-IRES2-eGFP (1)..(5859): Plasmid carrying the ITR-flanked construct of TASK-1 siRNA sequence 2 (directed against the human TASK-1 ortholog) coupled to an eGFP-Reporter via IRES2 under control of the cardiomyocyte specific CMV enhanced MLC260 promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgtctggaacatgacgagcgtgagttttggccactgactgactcacgctccatgttccagacaggacacaaggcctgttactagcactcacatggaacaaatggcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 5859 bp, Artificial Sequence
  Modified sequence
  pSSV9-CMV/MLC-miR/si-hTASK-1-3-IRES2-eGFP (1)..(5859): Plasmid carrying the ITR-flanked construct of TASK-1 siRNA sequence (directed against the human TASK-1 ortholog) coupled to an eGFP-Reporter via IRES2 under control of the cardiomyocyte specific CMV-enhanced MLC260 promoter
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttccttgtagttaatgattaacccgccatgctacttatctacgtagccatgctctagagcaagatcgaattcacgcgtcggcggccgcttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccatgcggccgctctaggacccagagcacagagcatcgttcccaggccaggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgcccaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgacggtaccgcggctggaggcttgctgaaggctgtatgctgagacgaagctgaaggccacgtgttttggccactgactgacacgtggcctagcttcgtctcaggacacaaggcctgttactagcactcacatggaacaaatggcggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgatgctagcctagtaataaaccggacattcgaaaggctgcggtcgaactctagttgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 16 - DNA, 21 bp, Artificial Sequence
  Modified sequence
  hTASK-1 (1)..(21): hTASK-1 siRNA from Hao and Li
ccttcagcttcgtgtacattt

SEQ ID NO: 17 - DNA, 21 bp, Artificial Sequence
  Modified sequence
  hTASK-1 (1)..(21): hTASK-1 siRNA from Olschewski et al.
tcaccgtcatcaccaccattt

SEQ ID NO: 18 - DNA, 21 bp, Artificial Sequence
  Modified sequence
  hTASK-1 (1)..(21): hTASK-1 siRNA from Gurney and Hunter (
aacaccttcgtgaggtacctt

SEQ ID NO: 19 - DNA, 21 bp, Artificial Sequence
  Modified sequence
  hTASK-1 (1)..(21): hTASK-1 siRNA from Tang et al.
tcaccgtcatcaccaccattt

SEQ ID NO: 20 - DNA, 60 bp, Homo sapiens
  humanTASK-1 (1)..(60): human TASK-1 sequence; coding sequence used for design of siRNA 1
cccagcacggatggcggcaaggtgttctgcatgttctacgcgctgctgggcatcccgctc

SEQ ID NO: 21 - DNA, 60 bp, Rattus norvegicus
  ratTASK-1 (1)..(60): rat TASK-1 sequence, coding sequence used for design of siRNA 1
cccagcacggacggaggcaaggtgttctgcatgttctacgcgctgctgggcatcccgctc

SEQ ID NO: 22 - DNA, 60 bp, Sus scrofa
  porcineTASK-1 (1)..(60): porcine TASK-1 sequence, coding sequence used for design of siRNA 1
cccagcacagacggcggcaaggtgttctgcatgttctacgcgctcttgggcatcccgctc

SEQ ID NO: 23 - DNA, 60 bp, homo sapiens
  humanTASK-1 (1)..(60): human TASK-1 sequence; coding sequence used for design of siRNA 2
ccgctcacgctcgtcatgttccagagcctgggcgagcgcatcaacaccttggtgaggtac

SEQ ID NO: 24 - DNA, 60 bp, Rattus norvegicus
  ratTASK-1 (1)..(60): rat TASK-1 sequence, coding sequence used for design of siRNA 2
ccgctcacactagtcatgttccagagcctgggtgaacgcatcaacaccttcgtgaggtac

SEQ ID NO: 25 - DNA, 60 bp, Sus scrofa
  porcineTASK-1 (1)..(60): porcine TASK-1 sequence, coding sequence used for design of siRNA 2
ccgctcacgctggtcatgttccagagcctgggcgagcgcatcaacaccttcgtgaagtac

SEQ ID NO: 26 - DNA, 60 bp, Homo sapiens
  humanTASK-1 (1)..(60): human TASK-1 sequence; coding sequence used for design of siRNA 3
gcagacgtggccttcagcttcgtctacatccttacgggcctcacggtcatcggcgccttc

SEQ ID NO: 27 - DNA, 60 bp, Rattus norvegicus
  ratTASK-1 (1)..(60): rat TASK-1 sequence, coding sequence used for design of siRNA 3
gcagatgtggccttcagcttcgtgtacatcctcacgggcctcacggtcatcggcgccttc

SEQ ID NO: 28 - DNA, 60 bp, Sus scrofa
  TASK-1porcine (1)..(60): porcine TASK-1 sequence, coding sequence used for design of siRNA 3
gcagacgtggcctttagcttcgtctacatcctcacgggcctcacggtcatcggcgccttt